(19) United States
(12) Patent Application Publication
(10) Pub. No.: US 2007/0004639 A1
(43) Pub. Date: Jan. 4, 2007

(54) Methods and compositions for treating Parkinson's disease

(75) Inventors: Patricia Kane, Millville, NJ (US); Edward Kane, Millville, NJ (US)

Correspondence Address: LAW OFFICES OF KHALILIAN SIRA, LLC, 9100 PERSIMMON TREE ROAD, POTOMAC, MD, 20854, US

(73) Assignee: BodyBio, Inc., Millville, NJ (US)

(21) Appl. No.: 11/171,308

(22) Filed: Jul. 1, 2005

(51) Int. Cl. A61K 38/05; A61K 31/685; A61K 31/202; A61K 31/19

(52) U.S. Cl. 514/18; 514/78; 514/557; 514/560

(57) ABSTRACT

The invention as disclosed herein provides pharmaneutical compositions and methods for treating, ameliorating, or preventing the symptoms of Parkinson's Disease. The pharmaneutical compositions of the invention contain in an effective amount a first and a second composition, the first composition comprises an effective amount of one or more phosphatidylcholine formulations and the second composition comprises an effective amount of one or more constituents comprising essential fatty acid supplements, trace minerals, butyrate, electrolytes, methylating agents, reduced glutathione, or a combination thereof, in a suitable carrier.

### FIELD OF THE INVENTION

This invention relates to the treatment of Parkinson's Disease with pharmaneutical compositions containing balanced essential nutritional supplements.

### I. BACKGROUND OF THE INVENTION

Parkinson's Disease, one of the two great neurodegenerative diseases of aging, is a progressive neurological disease affecting as many as 1,500,000 Americans. The other, Alzheimer's Disease, entails the progressive loss of memory and other mental difficulties. Parkinson's Disease occurs when certain nerve cells (neurons) in the part of the brain called the substantia nigra die or become impaired. Normally, these cells produce a vital chemical known as dopamine. Dopamine allows smooth, coordinated function of the body's muscles and movement. When approximately 80% of the dopamine-producing cells are damaged, the symptoms of Parkinson's Disease appear.

Parkinson's Disease affects both men and women in almost equal numbers. It shows no social, ethnic, economic or geographic boundaries. In the United States, it is estimated that 60,000 new cases are diagnosed each year. While the condition usually develops after the age of 65, 15% of those diagnosed are under 50 (Langston J W, 1995, The Case of the Frozen Addicts, Pantheon). Idiopathic Parkinson's Disease is by far the most common, and includes the rare genetic forms caused by mutations in the genes for alpha-synuclein and parkin. Known environmental causes include the very rare cases of poisoning by MPTP (1-methyl-4-phenyl-4-propionoxypiperidine), carbon monoxide, and manganese, as well as recurrent head trauma. Neuroleptic exposure, on the other hand, is a relatively common cause of drug-induced Parkinsonism (and is reversible).

The incidence of Parkinson's Disease increases with age. The median age of onset for all forms of Parkinson syndrome is 61.6 years, with median idiopathic Parkinson's Disease onset at 62.4 years. Onset before age 30 is rare, but up to 10% of cases of idiopathic Parkinson's Disease begin by age 40. In a recent study in the United States, the incidence of Parkinson's was 10.9 cases per 100,000 person years in the general population, and 49.7 per 100,000 person-years for these over age 50 (Bower, 1999). The incidence is growing as the population ages. Prevalence is estimated to be approximately 300 per 100,000 in the United States and Canada, but with the important caveat that perhaps 40% of cases may be undiagnosed at any given time.

Symptoms such as bradykinesia are slowness in voluntary movements. It produces difficulty initiating movement as well as difficulty completing movement once it is in progress. The delayed transmission from the brain to the skeletal muscles, due to diminished dopamine, produces bradykinesia. Tremors in the hands, fingers, forearm, or foot tend to occur when the limb is at rest, but not when performing tasks. Tremor may occur in the mouth and chin as well. Rigidity, or stiff muscles, may produce muscle pain and an expressionless, mask-like face. Rigidity tends to increase during movement. Poor balance is due to the impairment or loss of the reflexes that adjust posture in order to maintain balance. Falls are common in people with Parkinson's. The Parkinsonian gait is the distinctive unsteady walk associated with Parkinson's Disease. There is a tendency to lean unnaturally backward or forward, and to develop a stooped, head-down, shoulders-drooped stance. Arm swing is diminished or absent and people with Parkinson's tend to take small shuffling steps (called Destination). Someone with Parkinson's may have trouble starting to walk, appear to be falling forward as they walk, freeze in mid-stride, and have difficulty making a turn.

Parkinson's Disease symptoms may also include, micrographia (small hand writing), resting tremor, freezing episodes, painful leg cramps, akinesia—difficulty initiating movement, muscle stiffness, difficulty getting up from a chair, stooped over posture, facial masking, hypomimia—loss of facial expression, hypophonia—low voice volume, monotone speech, slurred, soft speech, staring, reduced blinking, eyelid apraxia, small shuffling steps, poor balance, rigidity—muscle, cogwheel rigidity—stop/start movements, drooling, seborrhea—unusually oily skin, fatigue easily, reduced arm swing, reduced ability to perform tasks such as handflipping and finger tapping, constipation, difficulty swallowing (dysphagia)—saliva and food that collects in the mouth or back of the throat may cause choking, coughing, or drooling, excessive salivation (hypersalivation), excessive sweating (hyperhidrosis), loss of bladder and/or bowel control (incontinence), loss of intellectual capacity (dementia)—late in the disease, slow response to questions (bradyphrenia) as well as psychosocial disorders such as, for example, anxiety, depression, and isolation.

There is no absolute cure for Parkinson's Disease up to date, however, there are a number of effective medicines that help to ease the symptoms of the disease.

1. Medications

1.1 Dopamine Agonists

Most symptoms are caused by lack of dopamine. The medicines most commonly used attempt to either replace or mimic dopamine, which improves the tremor, rigidity and slowness associated with Parkinson's Disease. Levodopa, a precursor to dopamine, was introduced as a Parkinson's Disease therapy in the 1960s, and remains the most effective therapy for motor symptoms. It alleviates most of cardinal motor symptoms of Parkinson's Disease, including bradykinesia, which is generally the most disabling feature of the disease.

Levodopa is a large neutral amino acid, which is absorbed in the gut and transported across the blood-brain barrier by the large neutral amino acid transporter. Thus, it competes with dietary amino acids for transport, and patients with advanced Parkinson's Disease may need to schedule the administration of their doses far from meal times, or they may reduce the protein content of their meals. Nausea and vomiting are the most common side effects, and are due to accumulation of dopamine in the blood stream (periphery). Orthostatic hypotension also occurs. The risk of hallucinations and paranoia increases over time, especially with advanced age. Compulsive behavior, including gambling and hypersexuality, is another risk (Marjama-Lyonns J 2003, Krivonos O 2004, Leiva C, Rev Neurol., 1997).

The toxic effects of levodopa are considerable. Low blood pressure is a common problem during the first few weeks, particularly if the initial dose is too high. In some cases the drug may cause abnormal heart rhythms. Stomach and intestinal side effects are common even with carbidopa. Levodopa can cause disturbances in breathing function, although it may benefit Parkinson's Disease patients who have upper airway obstruction. The mechanism of such actions is unclear. Drowsiness is a common adverse effect of levodopa and other dopaminergic therapies, and daytime somnolence and sudden sleep onset is possible. Patients may not experience any warning signs of sudden sleep onset; when such therapy is prescribed by a physician, patients need to be counseled and warned about the possibility of sudden sleep onset. In addition, patients should be reminded of the risk of sudden sleep onset when doses are increased or alternative medication is administered. No one agent appears to be more likely than others to cause these effects. The major adverse effects of the drug are psychiatric. Patients taking levodopa, especially in combination with other drugs, can experience confusion, extreme emotional states, anxiety, vivid dreams, effects on learning, sleepiness and sleep attacks (Maryland U. Medical Center).

The most troubling adverse effect from long-term levodopa use is dyskinesias, which typically begin to develop in milder form after three to five years of treatment, and become more severe after five to ten years of treatment. As the disease progresses, the dose required for symptomatic control approaches that which induces intolerable dyskinesias, thus narrowing the therapeutic window and limiting the continuing utility of levodopa. At this point, surgery may be the only effective option. Delaying commencement of levodopa therapy may be an appropriate strategy in younger patients. (Block, G., Liss, C., Reines, S., et al. Comparison of immediate-release and controlled-release carbidopa/levodopa in Parkinson's Disease: A multicenter 5-year study. Eur. Neurol 1997; 37:23-27).

Oral medications have been used to treat Parkinson's Disease and to replace, stimulate, or enhance dopamine activity in order to improve motor function. In order for these oral medications to work, they must first be absorbed by the gastrointestinal system and then cross the blood-brain barrier, where they can act on the dopamine brain cells. Since pure dopamine does not cross the blood-brain barrier, it must be delivered in the form of levodopa, which can cross into the brain. Early concern that levodopa may be neurotoxic in vivo does not seem to be borne out by clinical experience or recent research. Continuous duodenal infusion of levodopa is undergoing therapeutic trials as of mid-2004.

Levodopa continues to be the most effective treatment for motor symptoms, and all patients eventually require it. Long-term complications of dopaminergic therapy, however, are a concern that drives decision-making early in the treatment program.

The combination of levodopra with carbidopa (e.g., Sinemet) is the most potent medication for the treatment of Parkinson's Disease to date. Carbidopa, is an inhibitor of aromatic amino acid decarboxylation. Whereas in the past, levodopa was used alone, today it's known that carbidopa helps prevent the breakdown of levodopa so that it can effectively cross into the brain. Carbido/levodopa has been considered as an effective medication to control tremor, rigidity, and bradykinesia. (Cotzias G C, Papavasiliou P S, Gellen R. Modification of parkinsonism: chronic treatment with L-dopa. N. Engl. J. Med. 1969; 280:337-345).

In general, physicians and patients use the brand name Sinemet as a generic term, to refer to any carbidopa/levodopa drug. But there are many different forms and names for carbidopa/levodopa that can be prescribed, including Atamet and Sinemet E R, and they are considered relatively equivalent to Sinemet. Other drugs known as dopamine agonists include, for example, bromocriptine (Parlodel), pergolide (Permax), pramipexole (Mirapex), and ropinerole (Requip). These drugs have a similar chemical structure to dopamine and can cross the blood-brain barrier and directly stimulate the dopamine receptors.

Long-term use of carbidopa/levodopa, for example over five to ten years, is however associated with the development of motor complications in as many as 50 to 80 percent of Parkinson's Disease patients. The most disabling of these motor complications are the dyskinesias, involving irregular movements of the arms and legs and sometimes the face, neck, and trunk. At times the dyskinesias are severe and can be more disabling than the Parkinson's Disease symptoms themselves. Because of the side effect of dyskinesia with continued carbidopa/levodopa usage, some physicians try the dopamine agonist drugs first to delay the start of the use of carbidopa/levodopa. These drugs have demonstrated effectiveness in certain categories of Parkinson's Disease.

In one study, ropinerole (Requip) was shown to be as effective as levodopa in early stage Parkinson's Disease. Another study found Requip more effective than bromocriptine (Parlodel). In one study reported in the year 2000 in the New England Journal of Medicine, 268 Parkinson's patients were studied. Of that group, 179 were randomly selected to take ropinerole, and 89 received levodopa. After five years, among those patients taking ropinerole, only 20 percent developed dyskinesia, compared with 45 percent of those taking levodopa. Also, among those taking ropinerole who developed dyskinesia, only 8 percent had a severe form, versus 23 percent of those taking levodopa who developed dyskinesia. In another study, researchers from the Parkinson Study Group (PSG), a joint U.S. and Canadian organization, found that during the first two years, only 28 percent of patients who took pramipexole (Mirapex) developed motor complications, compared with 51 percent of patients who took levodopa. Starting treatment with pramipexole also appeared to delay the onset of motor complications. After two years, 72 percent of patients treated with pramipexole were completely free from motor complications. Dyskinesias developed in 31 percent of the levodopa patients but only 10 percent of the pramipexole patients. (Rascol O, Brooks D J, et al. Ropinerole reduces risk of dyskinesia compared to L-dopa when used in early Parkinson's Disease. Abstract presented at the International Congress on Parkinson's Disease in Vancouver; Jul. 24-28, 1999, Frucht S, Rogers J D, Greene P E, et al. Falling asleep at the wheel: motor vehicle mishaps in persons taking pramipexole and ropinerole. Neurology. 1999; 52:1908-1910.).

Carbidopa/levodopa has reported to have a shorter half-life than the dopamine agonists. For instance, a patient may have had good control of tremor and slower movements by taking Sinemet 25/100 (25 milligrams of carbidopa and 100 milligrams of levodopa) at 5 hours interval in the first 5 years from the initiation of the therapy. In the next 5 years, however, the same patient needs to take the medication every three hours for maximum effectiveness. Additionally, treatment with Carbidopa/levodopa may create an “on-off” phenomena, where one minute the medicine seems to be working and the next minute it would not work.

In several recent studies, dyskinesias occurred less often in patients treated with a dopamine agonist alone (5 percent) compared to levodopa alone (36 percent). In addition, patients treated with a dopamine agonist had less “off times,” periods when Parkinson's motor symptoms become disabling, compared to those treated with carbidopa/levodopa. The motor symptoms of tremor, rigidity, and bradylunesia were well controlled with dopamine agonists for up to five years in 30 percent of the patients, to such an extent that they did not need to add carbidopa/levodopa to their medication regimen. These recent findings support the use of dopamine agonists in newly diagnosed patients and in early mild-to-moderate Parkinson's Disease, then adding carbidopa/levodopa therapy when the patient's motor symptoms were not adequately controlled by dopamine agonists alone, or when intolerable side effects develop.

Despite the trend to use dopamine agonists as a first-line therapy to lessen the risk of developing dyskinesia, most people with Parkinson's Disease will need to add carbidopa/levodopa after three to five years to adequately control the motor symptoms. A recent study compared the effect of dopamine agonist bromocriptine to carbidopa/levodopa as the first medication used in the treatment of 782 persons with newly diagnosed untreated Parkinson's Disease. The study was conducted over ten years. The results showed only a slightly lower incidence of moderate to severe dyskinesia in the bromocriptine group. More importantly, the bromocriptine group had worsening motor function compared to the carbidopa/levodopa group, arguing that carbidopa/levodopa can be considered as a first-line therapy over dopamine agonists. In some cases, when side effects appear from carbidopa/levodopa therapy, the dose can be dropped down, and a dopamine agonist added in order to alleviate symptoms. One patient, was taking Sinemet CR 50/200 three times a day for two years with good control of his key symptoms—tremor and slowness—but then he lost the control of the movement of his head about two hours after he took his pill, which is quite common among Parkinson's patients (Pezzoli G, Martignoni E, Pacchetti C, et al. A crossover, controlled study comparing perolide with bromocriptine as an adjunct to levodopa for the treatment of Parkinson's Disease. Neurology. 1995; 45:S22-S27).

Parkinson's patients over 70 may be less tolerant of the dopamine agonist medications, due to side effects such as confusion, hallucinations, low blood pressure, nausea, vomiting, and daytime sleepiness. Similar side effects can occur with carbidopa/levodopa, but they tend to be less frequent than with the dopamine agonists. In general, there is no conclusive evidence for superior therapeutic activity of carbidopa/levodopa over dopamine agonists. Likewise, there is no evidence that any specific form of the drug, for example, the immediate release, controlled release (CR) and extended release (ER), may be superior to the other. Some doctors prefer one medicine to another, and some patients may respond better to one medicine than another, so to some extent, it is a trial-and-error process and there is no simple way to predict what medicine will work best or cause the least side effects.

As a general rule, the longer one has Parkinson's Disease, the more likely it is that one will be on multiple medications. Although a person might begin with a dopamine agonist, most people with Parkinson's Disease will eventually need to also be on carbidopa/levodopa to control the motor symptoms of Parkinson's Disease effectively and ultimately, many patients end up on a dopamine agonist in combination with carbidopa/levodopa and additional drugs.

Although carbidopa/levodopa and the dopamine agonists are the most effective medications for the treatment of the motor symptoms of Parkinson's Disease, several other classes of medications may be used on their own or in combination with these standard drugs. These drugs include, for example, Amantadine, the anticholinergics, MAOB (monoamine oxidase B) inhibitors, and the COMT (catecholamine-o-methytransferase) inhibitors, such as for example tolcapone (Tasmar) and entacapone (Comtan), help the carbidopa/levodopa function better by preventing the breakdown of levodopa, allowing more levodopa to cross the blood-brain barrier and act on dopamine neurons.

1.2. Anticholinergic

Another indication of Parkinson's Disease is the imbalance between acetylcholine and dopamine. Anticholinergic medications—such as trihexyphenidyl (Artane) and benztropine mesylate (Cogentin) are sometimes used in an effort to restore this balance, and help reduce tremor and rigidity in Parkinson's patients.

1.2.1. Amantadine

Amantadine is prescribed by its brand name Symmetrel, is an antiviral and Anticholinergic agent that has been used to treat the flu, and was found to help Parkinson's patients by reducing tremors, rigidity, and bradykinesia. Although its exact mechanism of action is unknown, it has been proposed that Amantadine may act as an N-Methyl-D-aspartate (NMDA) receptor antagonist. These NMDA receptor antagonists may protect dopamine brain cells from toxic damage, while also alleviating some of the symptoms of Parkinson's Disease. Amantadine, therefore, may have an added neuroprotective effect, protecting dopamine cells from injury. Amantadine was one of the first medications used to treat Parkinson's Disease and is considered to be a relatively weak drug compared to carbidopa/levodopa and the dopamine agonists, but it clearly does help to reduce Parkinson's Disease motor symptoms and recently has been shown to lessen dyskinesia. It can cause side effects similar to those of carbidopa/levodopa and the dopamine agonists, including nausea, vomiting, light-headedness, low blood pressure, anxiety, insomnia, confusion, and hallucinations. A rarer side effect, known as livedo reticularis, involves a purple-red mottled or marblelike appearance of the skin. In some patients, amantadine might work initially, but within weeks or months the benefits may stop. (Kornhuber, J., Weller M, Schoppmeyer K., and Riederer l, “Amantadine and memantadine are NMDA receptor antagonists with neuroprotective properties.” J Neural Transm 1994; 43(Suppl):S446).

Anticholinergics, like Amantadine, have been around a long time, and in fact were the first medications to be used for the treatment of Parkinson's Disease in the 1940s. The commonly prescribed drugs in the United States are trihexyphenidyl (Artane) and benztropine mesylate (Cogentin). These medicines have not been very effective in lessening bradykinesia (slowness), but do clearly help to lessen tremor and muscle rigidity and may reduce excessive drooling. However, they are not as beneficial as carbidopa/levodopa and the dopamine agonists and tend to cause more side effects, which limit their role in the treatment of Parkinson's Disease, especially in the elderly patient. Common side effects include confusion with or without hallucinations, urinary retention, blurry vision, dry mouth, hypotension, and constipation.

1.3. MAOB (Monoamine Oxidase B) Inhibitors

Selegiline, which is known by brand names Eldepryl and Deprenyl, is a monoamine oxidase (MAO) B inhibitor that can be used with carbidopa/levodopa to reduce motor fluctuations and increase “on” time. It should be prescribed at a 5-milligram dose two times daily, with the first dose taken on waking and the next dose taken not later than 2 P.M., in order to reduce the side effect of insomnia and vivid dreams. Various animal laboratory studies have shown a neuroprotective effect, meaning it prevents dopamine cells from injury and death from toxins, but it has not yet been proven to be a neuroprotective agent in human Parkinson's Disease patient studies. The DATATOP study in 1989, which involved 800 mild, early Parkinson's Disease patients, showed that Selegiline delays the progression of motor symptoms by about nine months, but this again is thought to happen because it enhances carbidopa/levodopa activity to control Parkinson's Disease motor symptoms.

Overall, Selegiline has a limited role in the treatment of Parkinson's Disease, as it helps improve motor symptoms only to a small degree, is not proven to slow down Parkinson's Disease, and can cause unwanted side effects. Some of the side effects, which are more common in the elderly, include insomnia, nightmares, hallucinations, and more rarely, heartburn, nausea, dizziness, loss of appetite, constipation, and worsened dyskinesia. (Olanow, C. W., and Calne, D. “Does selegiline monotherapy in Parkinson's Disease act by symptomatic or protective mechanisms?”, Neurology 1992; 42(Suppl 4): 13-26).

1.4. COMT (Catechol-O-Methyltransferase) Inhibitors

COMT inhibitors are relatively new drugs released in the late 1990s that prevent the breakdown of levodopa and thereby allow more levodopa to enter into the brain. Although similar to MAOB inhibitors that work by preventing the breakdown of dopamine by MAOB enzymes, COMT inhibitors (e.g., tolcapone) prevents the breakdown of both dopamine and L-dopa by COMT enzymes in both the central and peripheral nervous system (Entacapone cannot cross the blood-brain barrier, and only works peripherally). Administration of COMT inhibitors increases the availability of dopamine and L-dopa in the body, increases the concentration of dopamine, and increases the effectiveness of L-dopa treatments because the introduced L-dopa is not broken down before it reaches the brain. COMT enzymes that would break down the dopamine in the brain are also disabled by the inhibitor drug, and so dopamine successfully stimulates the necessary neurons to control motor functions that are inactive in patients with Parkinson's. Therefore, a higher percentage of the L-dopa administered to a patient is used by the body, and less of a dose is needed to maintain the same level of treatment. Decreasing the dosage often reduces the diskinesias associated with L-dopa.

The two available drugs in the family of COMT inhibitors are entacapone (Comtan) and tolcapone (Tasmar). These drugs should be used with carbidopa/levodopa to help to decrease “off”time by one to three hours a day and may allow for a lowering of the total daily dose of carbidopa/levodopa by 10 to 30 percent. It is important to know that the side effects of carbidopa/levodopa (dyskinesia, nausea, confusion, etc.) can occur or increase when a COMT inhibitor is added. Other side effects include blood in the urine (hematuria) in less than 1 percent of patients. These drugs can give a dark yellow-orange color to the urine, which is not harmful. Tasmar, but not Comtan, has also been linked to a very small chance of liver failure; it caused the death of three people with Parkinson's Disease in Europe, out of thousands of patients using the drug. Since those reports, Tasmar has been banned for use in Europe and is available for use in the United States, but with strict monitoring of liver function with routine blood testing. (The COMT inhibitor entacapone increases on time in Levodopa treated Parkinson 's Disease patients with motor fluctuations., Ann. Neurol. 1997; 46:747-755).

1.5. Botulinum Toxin

Botulinum toxin is a drug made from the bacteria that causes botulism, and is available in the United States in two forms, type A (Botox), which is the older of the two and has been studied for a variety of uses in Parkinson's Disease, and type B (Myobloc). It works by preventing the release of the chemical acetylcholine from the nerve at the neuromuscular junction. This chemical is needed to allow muscles to normally contract. When botulinum toxin is injected with a needle through the skin directly into the muscle, it causes the muscle to weaken and lessens the spasms or rigidity in the muscle. It takes three to five days after the injection before it begins to work, and the results last about two to three months before it wears off, requiring repeat injection. The use of botulinum toxin in the treatment of Parkinson's Disease is limited. It has been formally studied for the treatment of tremors by injecting it into the muscles of the arm that cause the tremor, but the results were not very promising. The botulinum toxin weakened hand muscles and reduced functional use of the limb, without any substantial reduction in tremors.

The drug has seemed more promising when treating selected leg or neck muscles, especially if the patient has only a few overactive muscles. Botulinum toxin may be helpful with foot dystonia, which involves cramping and painful turning of the foot that can make walking even more difficult for the Parkinson's Disease patient. It has also been used to treat excessive drooling by injecting the toxin directly into the salivary glands. It is considered to be a very safe drug in that it does not interact with other medications. The main side effect is weakening of the muscles that are injected (Henderson, J. M., Ghinka, J. A., Van Melle, G., et al. “Botulinum toxin A in non-dystonic tremors.” Eur. Neruol., 1996; 36:29-35; Ferrante, C., Perretti, A., Pomati, V., et al. “Botulinum toxin and Parkinson's Disease: A new therapeutical approach”., Abstract, Parkinsonism and Related Disorders, XIV International Congress on Parkinson's Disease, Helsinki, Finland, August 2001).

2. Surgery

Surgical treatment has become a mainstay of late-stage management, although not all patients can afford it or are appropriate candidates. From the 1940s through the 1960s, before the discovery of effective medications for the treatment of Parkinson's Disease, surgery of the brain was the primary treatment for Parkinson's Disease. In fact, tens of thousands of brain surgeries for Parkinson's Disease—known as thalamotomies and pallidotomies were performed in Europe and the United States. After levodopa was discovered by Dr. George Cotzias in 1967, the use of these surgical procedures declined dramatically, as the drug was a safer and less invasive alternative. In the past two decades, however, a renewed interest in surgical treatment of Parkinson's Disease has taken place (Kelly, l J., and Gillingham, F. J. “The long-term results of stereo-taxic surgery and L-dopa therapy in patients with Parkinson's Disease: A 10 year follow-up study.” J. Neurosurg., 1980; 53:322-327).

The main problem in the electrical pathway in the brain of a person with Parkinson's Disease is that the final motor circuit from the thalamus to the motor cortex is inhibited, or not working at full capacity. In order to enhance and restore positive electrical signals to stimulate the motor cortex to enable better movements, the pathways must be adjusted, much the way an electrician would fix an electrical short. This can be done in one of two ways: by creating a lesion or hole (similar to a small stroke) or by inserting a metal wire called an electrode, which is then turned on to electrically stimulate the motor circuit. Three main types of surgical treatments have been used for the treatment of Parkinson's Disease. These include the following:

2.1. Lesioning

Lesioning involves creating a small hole (‘btomy”) in the brain. Depending upon the location of the hole, different names are given to the procedure. For example, a lesion in the thalamus is called a thalamotomy, and a lesion in the globus pallidus is called a pallidotomy. (Lesioning of the subthalamic nucleus—subthalamotomy—has been found not to be an effective therapy).

Thalamotomy involves using a heat-sensitive probe to create a small hole in the thalamus of the brain. This technique is very effective at reducing tremor in Parkinson's Disease as well as essential tremor (not associated with Parkinson's Disease), by as much as 90 percent. Long-term benefit lasting up to ten years—has been reported in patients who have had a thalamotomy. Possible complications from the surgery include weakness or numbness on the opposite side of the body, partial visual loss, seizures, gait difficulty, slurred speech, and infection. Complications are fairly uncommon, however, and occur only in a small percentage of patients. In the case of thalamotomy and pallidotomy, the neurological symptoms may be permanent, as they result from brain tissue being destroyed during the procedure. Bilateral thalamotomy—lesioning of both the right and left thalamus is associated with a 30 percent risk of severe difficulty with speaking and swallowing, and since most experts agree that the risks far outweigh the benefits, this surgery is usually not performed (Burchiel, K. J., et al. “Comparison of pallidal and subthalamic nucleus deep brain stimulation for advanced Parkinson's Disease: Results of a randomized, blinded pilot study.” Neurosurg., 1999; 45: 13 75-1382).

Pallidotomy is similar to thalamotomy, except that the lesion is placed in a different part of the brain, the globus pallidus. Pallidotomy is by far the more commonly performed lesioning surgery. Only recently have clinical studies begun to document the effects of this procedure. Current data suggest that patients may benefit from this procedure, with a reduction in tremor, rigidity, bradykinesia, and off time by 15 to 50 percent at four months, and even up to four years after surgery. Some patients with tremor were shown to have a reduction of up to 75 percent, when using microelectrode recording (Fazzini E, Dogali M, Sterio C, et al. Stereotactic pallidotomy for Parkinson's Disease: a long term follow up of unilateral pallidotomy. Neurology, 1997; 49:665-67).

2.2. Electrical Stimulation

This involves placing a thin wire with an electrode at the end into the brain and then turning on the electrode to a battery source, and continuously stimulating the brain at a high frequency (100 to 180 hertz) to stimulate the brain motor pathways. The electrode may be placed at three different places in the brain: the thalamus, globus pallidus, or subthalamic nucleus. This procedure is called deep brain stimulation, or DBS for short. DBS is a relatively new technique pioneered by Dr. Alim-Louis Benabid in France in the late 1980s. Connecting the DBS of the thalamus results in a marked reduction of tremor in 92 percent of Parkinson's Disease patients, with results lasting up to eight years or more. However, similar to thalamotomy, the other motor symptoms of Parkinson's Disease—rigidity and bradykinesia are not greatly reduced by thalamic DBS. Bilateral DBS has been shown to be very effective in reducing the motor symptoms of Parkinson's Disease, without the risks of impaired mental functioning, swallowing, and speaking found in bilateral thalamotomy and pallidotomy.

There are reports that some patients who have had bilateral subthalarnic nucleus DBS are able to function independently without medications for Parkinson's Disease. A study reported in the New England Journal of Medicine compared bilateral DBS of the subthalamic nucleus to bilateral DBS of the globus pallidus sites in 134 patients with Parkinson's Disease. This study found that patients who received bilateral DBS to both sites had beneficial effects six months after surgery, with an increase in “on” time without dyskinesia from 27 to 74 percent in the subthalamic nucleus group and from 28 to 64 percent in the globus pallidus group. Nine patients had major adverse reactions—seven had intracranial hemorrhage and two had infection requiring that the electrodes be removed. Unlike pallidotomy, which is a onetime procedure, DBS requires that the electrodes be programmed and the battery pack or pulse generator—which is inserted under the skin of the chest—changed every two to five years. Serious side effects are seen in 2 percent of patients, and these can be permanent neurological deficits such as difficulty opening eyelids, weakness and numbness, and strokelike symptoms. Temporary, reversible complications include seizures, confusion, scalp or wound infection, electrode scalp erosion, numbness of the face or hand, and dyskinesia (“Electrical brain stimulation reduces Parkinson's symptoms,” American Academy of Neurology Online, http://www.aan.com/, Nov. 28, 2001).

2.3. Tissue Transplantation

Cell and/or tissue transplant therapies are expensive and still experimental, and their utility is currently compromised by the potential for unacceptable complications, which will require further preclinical work to both understand and avoid in the future. Transplantation involves taking some type of living tissue (from an aborted human fetus, from the fetus of an animal such as a pig, or from the patient) that contains dopamine cells and directly putting them into the brain of a patient with Parkinson's Disease. The results of tissue transplantation have not been particularly successful, however, and these procedures are considered to be experimental in comparison to the other types of surgery. Since the early 1980s, when adrenal gland transplantation was first performed, tissues that are rich in dopamine have been transplanted into the brains of patients suffering from advanced Parkinson's Disease. This procedure involved taking part of the patient's own adrenal gland and then putting it directly into the brain. Adrenal transplantation was not proven to be successful, and given its risks, it was quickly abandoned. Shortly afterward, human fetal brain cell transplants were introduced, and over two hundred Parkinson's Disease patients in several different countries had the procedure by the early 1990s. Results have been varied, mostly due to the variety of techniques. Those results, along with the ongoing ethical debate over using aborted human fetal tissue, and the higher cost of the procedure, have made tissue transplantation less popular, and it is considered experimental at best (Drucker-Colin R, Verdugo-Diaz L. Cell transplantation for Parkinson's Disease: present status. Cell Mol. Neurobiol., June 2004;24(3):301-16).

Unfortunately, none of the prior art therapies has yet been conclusively shown to slow or reverse the disease, although clinical trials of several candidates have shown intriguing results. Non-motor symptoms, especially depression, are increasingly being seen as important targets of therapy. Non-pharmacological treatments remain an important part of a comprehensive treatment program to-date.

The invention described herein solves the long felt need of treating, ameliorating, or preventing the symptoms of Parkinson's Disease and the long felt need of protecting individuals from developing symptoms of Parkinson's Disease by providing novel compositions and methods utilizing specific formulations and combination of different compositions that restore a healthy balance of essential nutrients paramount to maintain or restore the health of the individual and thereby preventing and healing the symptoms of Parkinson's Disease.

### II. SUMMARY OF THE INVENTION

The invention as disclosed herein provides pharmaneutical compositions and methods for treating or ameliorating the symptoms of Parkinson's Disease.

In one aspect, the invention provides pharmaneutical compositions comprising an effective amount of a first and a second composition, the first composition comprises one or more phosphotidylcholine formulations and the second composition comprises one or more constituents comprising essential fatty acid supplements, trace minerals, butyrate, electrolytes, methylating agent, reduced glutathione, or a combination thereof, in a suitable carrier.

In one embodiment, the first composition, the second composition, or both are formulated in one or different solutions, and/or they are in the same or different formulations, such as, for example in a liquid or dry formulation.

In another embodiment, the first composition, the second composition, or both are administered contemporaneously or at different time intervals.

In yet another embodiment, the first composition, the second composition, or both are administered in a time-released manner.

In another embodiment, the essential fatty acid supplements comprise linoleic acid and alpha linolenic acid in a ratio of about 4:1.

In yet another embodiment, the methylating agents comprise vitamin B compounds, such as, vitamin B12 and B complex compounds. These compounds include, for example, methylcobalamin, folinic acid compounds comprising Leucovorin, Citrovorum, Wellcovorin, or a combination thereof.

In another embodiment, the trace minerals comprise E-Lyte Liquid Mineral™ set #1-8 containing separate solutions of biologically available potassium, zinc, magnesium, copper, chromium, manganese, molybdenum, and selenium.

In yet another embodiment, the electrolytes comprise sodium, potassium, chloride, calcium, magnesium, bicarbonate, phosphate, and sulfate, or a combination thereof, among others.

In another aspect, the invention provides a method of treating, ameliorating, or preventing the symptoms of Parkinson's Disease in a subject, comprising administering to the subject an effective amount of a pharmanuetical composition comprising a first and a second composition, the first composition comprises one or more phosphatidylcholine formulations and the second composition comprises one or more constituents comprising essential fatty acid supplements, trace minerals, butyrate, electrolytes, methylating agent, reduced glutathione, or a combination thereof, in a suitable carrier or diluent, wherein the symptoms of Parkinson's Disease in the subject are treated, ameliorated, or prevented.

In yet another embodiment, the first composition, the second composition, or both is administered intravenously, orally, or both.

In another embodiment, about 500 mg to 1000 mg phosphatidylcholine is administered to the subject intravenously by lipid exchange twice to three times daily for about three to five days a week, and bolus amounts of phosphatidylcholine are used intravenously by IV drip as 7 grams to 21 grams one or more times monthly. About 3600 mg to about 18,000 mg of phosphatidylcholine is administered to the subject daily by mouth.

In another embodiment, about 910 mg to about 2600 mg of gamma linolenic acid contained in evening primrose oil is administered to the subject daily by mouth.

In yet another embodiment, about 30 mls to about 60 mls of the essential fatty acids (EFAs) 4:1 is administered to the subject daily by mouth.

In another embodiment, trace minerals are administered to the subject up to three times daily.

In another embodiment, oral electrolytes are administered to the subject up to five times daily.

In another embodiment, methylating agents folinic acid (tetrahydrofolate) as Leucovorin is administered to the subject intravenously as 5 mg (0.5 cc) to 10 mg (1 cc ) twice to three times daily for about three to five days a week in addition to daily injections as 1 cc or 25 mg of methylcobalamin.

In yet another embodiment, reduced glutathione is administered intravenously at about 1800 mg to about 2400 mg, 1-3 times daily, and for 2-4 days in a seven-day period and the subject is maintained on a low carbohydrate, high protein, and high fat diet.

In yet another embodiment, the invention provides a method of treating, ameliorating, or preventing the symptoms of Parkinson's Disease in a subject, comprising:

i) intravenous administration of a phosphatidylcholine composition comprising about 500 mg to 1000 mg phosphatidylcholine followed by intravenous administration of Leucovorin, Folinic Acid as 5 mg (0.5 cc) to 10 mg (1 cc ), and followed by intravenous administration of about 1800 mg to about 2400 mg of reduced glutathione, twice to three times daily for 3 to 5 days in a seven-day period; ii) once daily oral administration of a phosphatidylcholine composition comprising about 3600 to about 18,000 mg of phosphatidylcholine daily; iii) once or twice daily oral administration of an effective amount of one or more trace minerals; iv) once daily oral administration of about 30 mls to about 60 mls of an EFA 4:1 composition; v) once daily oral administration of about 910 mg to about 2600 mg of gamma linolenic acid in evening primrose oil; vi) oral administration of 1 oz oral electrolytes are administered up to five times daily and vii) once daily oral sublingual or injectable administration of 1 cc or 25 mg 3 to 7 times weekly of Methylcobalamin, wherein the subject is treated or the symptoms of Parkinson's Disease in the subject is treated, ameliorated, or prevented.

In yet another aspect, the invention provides a kit for the treatment, amelioration, or prevention of the symptoms of Parkinson's Disease in a subject, comprising: a) a first composition comprising one or more phosphatidylcholine formulations; b) a second composition comprising one or more constituents comprising: i) essential fatty acid supplements; ii) trace minerals; iii) butyrate or phenylbutyrate; iv) electrolytes; v) methylating agents folinic acid as Leucovorin and methylcobalamin; and vi) glutathione, c)instructions for the use of the first and second compositions; and d) instructions for where to obtain any missing components of the kit. The kit can further comprise instructions for determining an effective amount of the trace minerals for administration to the subject.

In one embodiment, the first composition, the second composition, or both are formulated in one or different solutions.

In another embodiment, the methods and compositions of the invention are used in combination with other commonly used treatments, medications, and/or surgical procedures for Parkinsons's Disease.

Other preferred embodiments of the invention will be apparent to one of ordinary skill in the art in light of what is known in the art, in light of the following description of the invention, and in light of the claims.

### III. DETAILED DESCRIPTION OF THE INVENTION

The invention as described herein provides pharmaneutical compositions and methods for treating, ameliorating and/or preventing the symptoms of Parkinson's Disease and inhibiting the progression of the disease using a composition containing nutritional supplements. The invention also provides methods of treating a subject at risk for developing Parkinson's Disease in order to delay the onset of Parkinson's Disease symptoms.

The pharmaneutical compositions and methods of the invention are designed on the principle of “balanced nutrients” and “stabilization of phospholipids within the cell membrane”. The normal body keeps a healthy balance among essential nutrients that is a key in the well being and health of the individual. Unlike most therapies that cause an imbalance in the body of a sick individual who is already comprised by the sickness or the disease itself, the compositions and therapeutic methods of the present invention heal the subject individual by restoring the balance of essential nutrients to adjust it to a normal level in order to assist the body to fight the abnormal condition and/or ailments and to increase the ability of the immune system to fight the disease.

As used herein, a “pharmaneutical composition” includes any composition in which at least 50% of its compounds, compositions and/or constituents have been derived from natural sources and/or are used in their natural form, as opposed to being chemically, or synthetically produced.

As used herein, a “subject” is any mammal, in particular a primate, preferably a human, that 1) exhibits at least one symptom associated with Parkinson's Disease; 2) has been diagnosed with Parkinson's Disease; or 3) is at risk for developing Parkinson's Disease.

As used herein, a “subject at risk for developing Parkinson's Disease” includes subjects with a family history of Parkinson's Disease or who are susceptible to developing Parkinson's Disease. Subjects “susceptible to developing Parkinson's Disease” include those subjects testing positive for molecular markers indicative of or associated with Parkinson's Disease. However, some patients can find that getting a diagnosis of Parkinson's Disease is a challenge. There are no diagnostic tests for Parkinson's Disease, meaning that a brain scan does not diagnose it. The dopamine cells that die off in Parkinson's Disease are in such a small area of the brain that a CT scan or MRI of the brain is not able to show theses microscopic changes, and most patients with Parkinson's Disease will have normal brain scans.

As used herein, an “effective amount” of a composition is an amount sufficient to achieve a desired biological effect, in this case at least one of prevention, amelioration or treatment of Parkinson's Disease. It is understood that the effective dosage will be dependent upon the age, sex, health, and weight of the recipient, kind of concurrent treatment, if any, frequency of treatment, and the nature of the effect desired. The most preferred dosage will be tailored to the individual subject, as is understood and determinable by one of skill in the art, without undue experimentation.

As used herein, a “carrier” refers to a diluent, adjuvant, excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Sterile water is a preferred carrier when the pharmanuetical composition is administered intravenously. Saline solutions and aqueous dextrose and glycerol solutions can also be employed as liquid carriers, particularly for injectable solutions.

As used herein, Glutathione, and rGlutathione (Reduced Glutathione) are used interchangeably herein.

1. Description of Pharmaneutical Constituents

1.1 Phosphatidylcholine

Phosphatidylcholine (PC) is the predominant phospholipid of all cell membranes and of the circulating blood lipoproteins. PC is the main lipid constituent of the lipoprotein particles circulating in the blood and the preferred precursor for certain phospholipids and other biologically important molecules. PC also provides antioxidant protection in vivo. In animal and human studies, PC protected against a variety of chemical toxins and pharmaceutical adverse effects.

Chemically, PC is a glycerophospholipid that is built on glycerol (CH2OH—CHOH—CH2OH) and substituted at all three carbons. Carbons 1 and 2 are substituted by fatty acids and carbon 3 by phosphorylcholine. Simplistically, the PC molecule consists of a head-group (phosphorylcholine), a middle piece (glycerol), and two tails (the fatty acids, which vary). Variations in the fatty acids in the tails account for the great variety of PC molecular species in human tissues.

In vivo, PC is produced via two major pathways. In the predominant pathway, two fatty acids (acyl “tails”) are added to glycerol phosphate (the “middle piece”), to generate phosphatidic acid (PA) that is converted to diacylglycerol, after which phosphocholine (the “head-group”) is added on from CDP-choline. The second, minor pathway is phosphatidylethanolamine (PE) methylation, in which the phospholipid PE has three methyl groups added to its ethanolamine head-group, thereby converting it into PC.

Taken orally PC is very well absorbed, up to 90% per 24 hrs when take with meals. PC enters the blood gradually and its levels peak over 8-12 hours. During the digestive process, the position-2 fatty acid becomes detached (de-acylation) in the majority of the PC molecules. The resulting lyso-PC readily enters intestinal lining cells, and is subsequently re-acylated at this position. The position-2 fatty acid contributes to membrane fluidity (along with position I), but is preferentially available for eicosanoid generation and signal transduction. The omega-6/omega-3 balance of the PC fatty acids is subject to adjustment via dietary fatty acid intake. Choline is most likely an essential nutrient for humans, and dietary choline is ingested predominantly as PC. Greater than 98 percent of blood and tissue choline is sequestered in PC that serves as a “slow-release” blood choline source.

Methyl group (—CH3) availability is crucial for protein and nucleic acid synthesis and regulation, phase-two hepatic detoxification, and numerous other biochemical processes involving methyl donation. Methyl deficiency induced by restricted choline intake is linked to liver steatosis in humans, and to increased cancer risk in many mammals. PC is an excellent source of methyl groups, supplying up to three per PC molecule, and is the main structural support of cell membranes, the dynamic molecular sheets on which most life processes occur. Comprising 40 percent of total membrane phospholipids, PC's presence is important for homeostatic regulation of membrane fluidity. PC molecules of the outermost cell membrane deliver fatty acids on demand for prostaglandin/eicosanoid cellular messenger functions, and support signal transduction from the cell's exterior to its interior.

PC compositions used within the scope of the invention include, by way of example and not limitation, compositions comprising phosphatidylcholine including Essentiale N™ or LipoStabil™ 500 mg to 1000 mg phosphatidylcholine used intravenously by lipid exchange or in a bolus IV solution as 7 grams to 21 grams, available from A. Natterman & Cie, GmbH (Cologne, Germany); PhosChol™ 100% phosphatidylcholine preparation available from Nutrasal™ LLC (Oxford, Conn. USA), and from BodyBio Inc. (Millville, N.J. USA).

1.2 Essential Fatty Acids (EFAs)

Essential Fatty Acids (EFAs) are long-chain polyunsaturated fatty acids derived from linolenic, linoleic, and oleic acids. EFAs are necessary fats that humans cannot synthesize, and must be obtained through diet. EFAs compete with undesirable fats (e.g., trans fats and cholesterol) for metabolism. Also, EFAs raise the HDL (High Density Lipoprotein) that is also considered beneficial for the body by capturing the undesirable LDL (Low Density Lipoprotein), and escort it to the liver where it is broken down and excreted.

There are two families of EFAs: Omega-3 and Omega-6. Omega-9 is necessary yet “non-essential” because the body can manufacture it in a modest amount, provided essential EFAs are present. The number following “Omega-” represents the position of the first double bond, counting from the terminal methyl group on the molecule. Omega-3 fatty acids are derived from Linolenic Acid, Omega-6 from Linoleic Acid, and Omega-9 from Oleic Acid.

EFAs support the cardiovascular, reproductive, immune, and nervous systems. The human body needs EFAs to manufacture and repair cell membranes, enabling the cells to obtain optimum nutrition and expel harmful waste products. A primary function of EFAs is the production of prostaglandins, which regulate body functions such as heart rate, blood pressure, blood clotting, fertility, conception, and play a role in immune function by regulating inflammation and encouraging the body to fight infection. Essential Fatty Acids are also needed for proper growth in children, particularly for neural development and maturation of sensory systems, with male children having higher needs than females. Fetuses and breast-fed infants also require an adequate supply of EFAs through the mother's dietary intake. Because high heat destroys linolenic acid, cooking in linolenic-rich oils or eating cooked linolenic-rich fish is unlikely to provide a sufficient amount.

EFA deficiency is common in the United States, particularly Omega-3 deficiency and now Omega-6 deficiency due to the increased use of hydrogenated vegetable oil, and recently, over prescribing and consumption of Fish Oil. Essential fatty acid supplements include solutions comprising a mixture of omega 6 and omega 3 fatty acids, in ratio of from about 20/1, 10/1, 5/1, 4/1, 3/1, 2/1, 1/1, or less. It is intended herein that by recitation of such specified ranges, the ranges recited also include all those specific integer amounts between the recited ranges. For example, in the range of about 4/1, it is intended to also encompass 4.2/1, 3.8/1, 3.5/1, 3.2/1, 3/1, etc, without actually reciting each specific range therewith. Preferably the ratio between the omega 6 and omega 3 fatty acids is about 4/1 v/v.

1.2.1 Omega-3 Fatty Acids

Alpha Linolenic Acid (ALA) is the principal Omega-3 fatty acid, which a healthy human will convert into eicosapentaenoic acid (EPA), and later into docosahexaenoic acid (DHA). Omega-3s are used in the formation of cell walls, making them supple and flexible, and improving circulation and oxygen uptake with proper red blood cell flexibility and function.

Omega-3 deficiencies are linked to decreased memory and mental abilities, tingling sensation of the nerves, poor vision, increased tendency to form blood clots, diminished immune function, increased triglycerides and “bad” cholesterol (LDL) levels, impaired membrane function, hypertension, irregular heart beat, learning disorders, menopausal discomfort, and growth retardation in infants, children, and pregnant women.

Food containing alpha linolenic acid includes flaxseed oil, flaxseed, flaxseed meal, hempseed oil, hempseed, walnuts, pumpkin seeds, Brazilian nuts, sesame seeds, avocados, some dark leafy green vegetables (e.g., kale, spinach, mustard greens, collards, etc.), canola oil (cold-pressed and unrefined), soybean oil, and others. Higher order omega 3 fatty acids (HUFA) include wild salmon, mackerel, sardines, anchovies, albacore tuna, cod liver oil, fish oil, and other cold water fish. Foods rich in higher order —HUFA omega-3 fatty acids—as wild salmon and sardines are suggested to the subjects as part of their diet.

In one embodiment, One part of alpha linolenic acid as cold pressed, organic flaxseed oil is utilized with four parts of linoleic acid omega-6 oil as cold pressed, organic sunflower oil as a 4:1 omega-6 to omega 3 ratio balanced oil.

1.2.2. Omega-6 (Linoleic Acid)

Linoleic Acid is the primary Omega-6 fatty acid. A healthy human with good nutrition will convert linoleic acid into gamma linolenic acid (GLA), which will later synthesized with EPA from the Omega-3 group into eicosanoids. Eicosanoids are hormone-like compounds, which aid in many bodily functions including vital organ function and intracellular activity.

Some Omega-6s improve diabetic neuropathy, rheumatoid arthritis, PMS, skin disorders (e.g. psoriasis and eczema), inflammation, allergies, autoimmune conditions and aid in cancer treatment.

Food containing linoleic acid includes safflower oil, sunflower seed, sunflower oil, hempseed oil, hempseed, pumpkin seeds, borage oil, evening primrose oil, black currant seed oil, among many others. In one embodiment, evening primrose oil is utilized daily as part of the therapy for Parkinson's as about 910 mg to about 2600 mg of gamma linolenic acid is contained in this oil. In another embodiment, four parts of linoleic acid omega-6 oil as cold pressed, organic sunflower oil is utilized along with 1 part of alpha linolenic acid as cold pressed, organic flaxseed oil as a 4:1 omega 6 to omega 3 ratio balanced oil.

1.3. Methylating Agents

Methylating agents donate methyl groups to molecules to enhance or reduce their expression. One important function of Methylating agents is in cellular regeneration and repair per stimulation of DNA expression. Another important function of methylating agents is to selectively “rescue” normal cells from the adverse effects of methotrexate or other poisonous substances. Other functions of methylating agents involve impeding the ability of cancer cells to divide.

Encompassed within the scope of the claimed invention are several types and classes of methylating agents. In a preferred embodiment of the invention, the methylating agent is in a natural form or derived from a natural source. Such natural methylating agents include, by way of example and not limitation, agents within the family of vitamin B group of vitamins including Methylcobalamin, Leucovorin/Folinic Acid, or a combination thereof.

Disturbances in methylation pathways may occur after exposure to heavy metals, thimerosal (preservative in vaccinations), large quantities of alcohol, or chemicals or medication (terbutaline). See, for example, in MOLECULAR ORIGINS OF HUMAN ATTENTION—THE DOPAMINE—FOLATE CONNECTION by Richard C. Deth (Kluwer Academic Publishers: Norwell, Mass., (2003)), incorporated herein by reference in its entirety. Dr. Deth, describes damage to the enzyme methionine synthase after exposure to heavy metals and alcohol whereby the enzyme may be stimulated by the use of the methylated B vitamins methylcobalamin and tetrahydrofolate or folinic acid. A direct connection between polymorphism resulted from toxic exposures to the enzyme methylene tetrahydrofolate reductase (MTHFR) has also been widely documented in the literature. If methylation pathways are not supported with methylated forms of the B vitamins folinic acid and methylcoblamin, the ability to detoxify, balance hormones, stabilize cell membrane functions, rejuvenate DNA expression, and to lock neurotransmitters such as dopamine and serotonin to their receptors is grossly impaired.

1.3.1. Methylcobalamin

Methylcobalamin is a type of Vitamin B12. Vitamin B12 has several different formulations including hydroxy, cyano, and adenosyl, but only the methyl form is used in the central nervous system. Deficiency states are fairly common and vitamin B12 deficiency mimics many other disease states of a neurological or psychological kind, and it causes anemia. B12 is converted by the liver into methylcobalamin but not in therapeutically significant amounts. Vitamin B12 deficiency is caused by a wide range of factors including low gastric acidity (common in older people) use of acid blockers such as Prilosec™ or excessive laxative use, lack of intrinsic factor, poor absorption from the intestines, lack of Calcium, heavy metal toxicity, excessive Vitamin B12 degradation, internal bleeding, excessive menstrual flow, exposure to high amounts of alcohol, or damage to methylation pathways/enzymes such as methylene tetrahydrofolate reductase (MTHFR) due to toxicity exposure.

Methylcobalamin donates methyl groups to the myelin sheath that insulates nerve fibers and regenerates damaged neurons. In a B12 deficiency, toxic fatty acids destroy the myelin sheath but high enough doses of B12 can repair it. Methylcobalamin is better absorbed and retained than other forms of B12 (such as cyanocobalamin). Methylcobalamin protects nerve tissue and brain cells and promotes healthy sleep and is a cofactor of methionine synthase, which reduces toxic homocysteine to the essential amino acid methionine. Methylcobalamin also protects eye function against toxicity caused by excess glutamate.

1.3.2. Leucovorin, Tetrahydrofolate, Folinic Acid

Leucovorin is the active form of the B complex vitamin, tetrahydrofolate. Leucovorin is used as an antidote to drugs that decrease levels of Folinic Acid. Folinic Acid assists the formation of red and white blood cell and the synthesis of hemoglobin. Some treatments require what is called leucovorin rescue, because the drug used to treat the cancer or other infection has had an adverse effect on Folinic Acid levels. Leucovorin is used to reduce anemia in people taking dapsone. Leucovorin is also taken to decrease the bone marrow toxicity of sulfa drugs, and in combination with pyrimethamine to decrease the toxicity of toxoplasmosis treatment. Leucovorin is also used in combination with trimetrexate to prevent bone marrow toxicity and in combination with chemotherapeutic agents such as methotrexate. Other substituents for Leucovorin include Citrovorum, Wellcovorin, and/or folinic acid, among others.

Leucovorin calcium (folinic acid) is a reduced form of folic acid. It is usually used 24 hours after methotrexate to selectively “rescue” normal cells from the adverse effects of methotrexate caused by inhibition of production of reduced folates. It is not used simultaneously with methotrexate, as it might then nullify the therapeutic effect of the methotrexate. More recently, leucovorin has also been used to enhance the activity of fluorouracil by stabilizing the bond of the active metabolite (5-FdUMP) to the enzyme thymidylate synthetase. Commercially available Leucovorin is the racemic mixture of D and L isomers. It is now recognized that the activity of Leucovorin is due to the L form.

1.3.3. Synthetic Methylating Agents

Synthetic methylating agents, which impair the ability of malignant cells to divide, include dacarbazine (DTIC), temozolomide (TMZ), procarbazine, Methylnitrosourea, N-methyl-N-nitrosourea (MNU), methyl methanesulfonate (MMS) and methyl iodide, among others.

1.4 Glutathione

Reduced Glutathione (rGlutathione) is known chemically as N—(N-L-gamma-glutamyl-L-cysteinyl)glycine and is abbreviated as GSH. Its molecular formula is C10H17N3O6S and its molecular weight is 307.33 Daltons. Glutathione disulfide is also known as L-gamma-glutamyl-L-cysteinyl-glycine disulfide and is abbreviated as GSSG. Its molecular formula is C20H32N6O12S2. The term glutathione is typically used as a collective term to refer to the tripeptide L-gamma-glutamyl-L-cysteinylglycine in both its reduced and dimeric forms. Monomeric glutathione is also known as reduced glutathione and its dimer is also known as oxidized glutathione, glutathione disulfide and diglutathione. Reduced glutathione is also called glutathione and the glutathione dimer is referred to as glutathione disulfide.

Glutathione is widely found in all forms of life and plays an essential role in the health of organisms, particularly aerobic organisms. In animals, including humans, and in plants, glutathione is the predominant non-protein thiol and functions as a redox buffer, keeping with its own SH groups proteins in a reduced condition, among other antioxidant activities.

Glutathione plays roles in catalysis, metabolism, signal transduction, gene expression and apoptosis. It is a cofactor for glutathione S-transferases, enzymes which are involved in the detoxification of xenobiotics, including carcinogenic genotoxicants, and for the glutathione peroxidases, crucial selenium-containing antioxidant enzymes. It is also involved in the regeneration of ascorbate from its oxidized form, dehydroascorbate.

Glutathione functions as an antitoxin as well as antioxidant and is extremely important for the protection of major organs, the function of the immune system, and the fight against aging. It minimizes the damage caused by free radicals that is important for the health of cells. Recent, extensive research has shown the direct relationship between decreased glutathione levels and the progression of many chronic diseases. It is reported that decreased Glutathione may be a result of various types of prolonged stress and hyperactivity of the immune system, which in turn compromises the health of the body's cells. Unfortunately, taking Glutathione (L-Glutathione capsules) orally is not a suitable method for replacement of losses since the glutathione molecule is very unstable and is destroyed by the stomach acid before it can be absorbed.

Gluthathione's major effect is intracellular, and intra-organelle. Within the mitochondria Glutathione is present in tissues in concentrations as high as one millimolar. There are undoubtedly roles of glutathione that are still to be discovered.

1.5 Butyrate, Sodium Phenylbutyrate

Butyrate is an important short chain fatty acid that provides fuel for colon cells and may help protect against colon cancer. The most potent dietary source of butyrate is reported to be butter (3%). Butyrate is made in the colon by bacteria. Antibiotics kill the bacteria that produce butyrate. Butyrate has a particularly important role in the colon, where it is the preferred substrate for energy generation by colonic cells.

Butyrate has been shown to significantly inhibit the growth of cancerous colon cells. Scientists have found a human gene that stops the growth of cancer cells when activated by fiber processing in the colon. Whether by supplement or by enema, a few pilot studies suggest that the presence of butyrate in colon is useful in reducing symptoms and restoring indicators of colon health in ulcerative colitis, but one study showed no benefit over placebo. Several doctors claim that many people are helped with butyrate enemas. Butyrate levels are commonly measured in comprehensive stool analyses and act as a marker for levels of beneficial bacteria.

Excess of butyrate in the body harms cellular functions. On possible mechanism of action of butyrate is through breaking up ceramides which accumulate in the membrane as clusters called “lipid rafts”. Rafts are composed of ceramides, cholesterol and sphingomyelin (SM) all of low energy with either very long chains or rigid chains (e.g. cholesterol.) Ceramides are generally structured with lipid tails as very long chain fatty acids (VLCFAs) and combine with PC to form SM (reversible back into ceramide and phosphatidylcholine). SM maintains the VLCFAs from the ceramide as opposed to holding on to the former high active lipids formerly associated with PC. Most diseases and aging tends towards a higher concentration of raft formation. This is complicated with signaling emanating from rafts that encourages apoptosis, which is both destructive and constructive.

The low activity level of the three lipids encourages the agglomeration into rafts which ultimately degrades the fluidity of vibrant active membranes. Most diseases and aging tend towards a higher concentration of raft formation. This is complicated with signaling emanating from rafts that encourage apoptosis, which is both destructive and constructive.

Although scientists have long linked butyrate to overall reductions in the incidence of colon cancer, the molecular basis of that benefit has remained largely unknown. Butyrate affects a chemical that otherwise bind and constrict the activity of the p21 gene that is involved in the growth of cancer cells. Butyrate optimizes itself in the body. Concentrations of butyrate in the composition of the invention can range from about 1-10 grams per liter or more, depending on the specific condition at hand. Minamiyama et al. Hum. Mol. Genet. 1;13(11):1183-92. Epub (2004) (incorporated herein by reference by its entirety) in a study using mouse model of Bulbar ALS, demonstrated oral administration of sodium butyrate (SB) successfully ameliorated neurological phenotypes as well as increased acetylation of nuclear histone in neural tissues.

1.6 Electrolytes

Electrolyte is a “medical/scientific” term for salts, specifically ions. The term electrolyte means that ion is electrically-charged and moves to either a negative (cathode) or positive (anode) electrode. Electrolytes are vital elements of a healthy body and are needed for the proper performance of bodily organs and tissues by maintaining the voltages across the cell membranes and to carry electrical impulses (nerve impulses, muscle contractions) across these cells and to other cells. The kidneys function is to keep the electrolyte concentrations in the blood constant despite changes in the body. For example, during a heavy exercise the body loses electrolytes in the sweat, particularly sodium and potassium. These electrolytes must be replaced to keep the electrolyte concentrations of the body fluids constant. So, many sports drinks have sodium chloride or potassium chloride added therein.

The types of electrolytes used within the scope of the invention include, by way of example and not limitation, sodium (Na⁺), potassium (K⁺), chloride (Cl⁻), Calcium (Ca²), Magnesium (mg²), bicarbonate (HCO₃⁻), Phosphate (PO₄⁻²) and sulfate (SO₄⁻²), among others.

1.7 Trace Minerals

Another important constituent of the pharmaneutical composition of the invention as described herein includes trace minerals. Suitable mineral compositions include solid multi-mineral preparations, or the E-Lyte Liquid Mineral™ set #1-8 (separate solutions of biologically available potassium, zinc, magnesium, copper, chromium, manganese, molybdenum, and selenium, or a combination thereof, or #1-9 (separate solutions of biologically available potassium, zinc, magnesium, copper, chromium, manganese, molybdenum, selenium and iodine), or a combination thereof. Both E-Lyte Liquid Mineral™ set #1-8, and E-Lyte Liquid Mineral™ set #1-9 set are available from E-Lyte, Inc. (Millville, N.J., USA).

2. Pharmaneutical Compositions

The present invention provides pharmaneutical compositions comprising a therapeutically effective amount of a first composition comprising one or more phosphotidylcholine formulations and the second composition comprising one or more constituents comprising essential fatty acid supplements, trace minerals, butyrate, electrolytes, methylating agents (methylcobalamin, folinic acid/Leucovorin), glutathione, or a combination thereof, in a suitable carrier.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In general, the combinations may be administered by the transdermal, intraperitoneal, intracranial, intracerebroventricular, intracerebral, intravaginal, intrauterine, oral, rectal, ophthalmic (including intravitreal or intracameral), nasal, topical (including buccal and sublingual), parenteral (including subcutaneous, intraperitoneal, intramuscular, intravenous, intradermal, intracranial, intratracheal, and epidural) administration.

A typical regimen for preventing, suppressing, or treating Parkinson Disease comprises administration of an effective amount of the composition as described above, administered as a single treatment, or repeated as enhancing or booster dosages, over a period up to and including one week to about 48 months or more.

The pharmaneutical compositions of the present invention, suitable for inoculation or for parenteral or oral administration, are in the form of sterile aqueous or non-aqueous solutions, suspensions, or emulsions, and can also contain auxiliary agents or excipients that are known in the art.

In one embodiment, the composition is formulated in accordance with routine procedures adapted for intravenous administration to human beings. Typically, compositions for intravenous administration are solutions in sterile isotonic aqueous buffer. Where necessary, the composition may also include a solubilizing agent and a local anesthetic such as procaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water (not saline). Where the composition is administered by injection, an ampoule of sterile water for injection or saline can be provided so that the ingredients may be mixed prior to administration.

In addition, the compositions of the invention may be incorporated into biodegradable polymers allowing for sustained release of the compound, the polymers being implanted in the vicinity of where the delivery is desired, so that the composition is slowly released systemically.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaneutical composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Within other embodiments, the compositions may also be placed in any location such that the compounds or constituents are continuously released into the aqueous humor. The amount of the composition of the invention which will be effective in the treatment, inhibition and prevention of Parkinson's Disease can be determined by standard clinical techniques. In addition, in vitro assays may optionally be employed to help identify optimal dosage ranges.

In particular, the dosage of the compositions of the present invention will depend on the disease state of Parkinson's Disease and other clinical factors such as weight and condition of the human or animal and the route of administration of the compounds or compositions. The precise dose to be employed in the formulation, therefore, should be decided according to the judgment of the health care practitioner and each patient's circumstances. Effective doses may be extrapolated from dose-response curves derived from in vitro or animal model test systems.

Treating humans or animals between approximately 0.5 to 500 mg/kilogram is a typical broad range for administering the pharmaneutical composition of the invention. The methods of the present invention contemplate single as well as multiple administrations, given either simultaneously or over an extended period of time.

Preferred unit dosage formulations are those containing a daily dose or unit, daily sub-dose, or an appropriate fraction thereof, of the administered compositions. It should be understood that in addition to the compositions, particularly mentioned above, the formulations of the present invention may include other agents conventional in the art having regard to the type of formulation in question.

The pharmaneutical composition of the invention comprises a dry formulation, an aqueous solution, or both. Effective amounts of a phosphatidylcholine composition, EFA composition, trace minerals, rGlutathione, butyrate, electrolytes, or methylating agents (methylcobalamin, Leucovorin/folinic acid) can each be formulated into the pharmaneutical composition for treating Parkinson's Disease or for delaying the onset of Parkinson's Disease symptoms in a subject. As used herein, a “pharmaneutical composition” includes compositions for human and veterinary use. Pharmaneutical compositions for parenteral (e.g., intravascular) administration are characterized as being sterile and pyrogen-free. One skilled in the art can readily prepare pharmaneutical compositions of the invention for enteral or parenteral use, for example by using the principles set forth in Remington's Pharmaceutical Science, 18^{th} edit. (Alphonso Gennaro, ed.), Mack Publishing Co., Easton, Pa., 1990.

Because phosphatidylcholine, linoleic acid and alpha linolenic acid are all soluble in oils or lipids, they can be conveniently formulated into a single pharmaneutical composition. Thus, in one embodiment, the invention provides a single-dose pharmaneutical composition comprising a phosphotidylcholine composition and an EFA 4:1 composition. Those constituents that are water soluble, such as for example, the liquid trace minerals, and electrolytes are generally not formulated into a single pharmaneutical composition with the phosphatidylcholine and EFAs compositions, but are rather formulated as separate compositions. However, the water soluble constituents, the phosphatidylcholine composition, and the EFA composition can be formulated into a single pharmaceutical composition as an emulsion, for example an oil-in-water emulsion or water-in-oil emulsion.

The pharmaneutical compositions of the invention can be in a form suitable for oral use, according to any technique suitable for the manufacture of oral pharmaceutical compositions as are within the skill in the art. For example, the phosphatidylcholine composition and the EFA composition can be formulated (either separately or together) into soft capsules, oily suspensions, or emulsions, optionally in admixture with pharmaceutically acceptable excipients. Suitable excipients for a phosphatidylcholine composition or EFA composition comprise oil-based media; e.g., archis oil, liquid paraffin, or vegetable oils such as olive oil. Butyrate is administered in encapsulated form, for example, as Magnesium/Calcium Butyrate from BodyBio, Inc., (Millville, N.J., USA) or Sodium Phenylbutyrate from Triple Crown America (Perkasie, Pa., USA) or as IV Liquid Sodium PhenylButyrate from Medaus Pharmacy (Birmingham, Ala., USA).

The compositions of the invention are formulated into liquid or solid compositions, such as aqueous solutions, aqueous or oily suspensions, syrups or elixirs, emulsions, tablets, dispersible powders or granules, hard or soft capsules, optionally in admixture with pharmaceutically acceptable excipients.

2.1. Adjuvants, Carriers, and Diluents

As would be understood by one of ordinary skill in the art, when a composition of the present invention is provided to an individual, it can further comprise at least one of salts, buffers, adjuvants, or other substances which are desirable for improving the efficacy of the composition. Adjuvants are substances that can be used to specifically augment at least one immune response. Normally, the adjuvant and the composition are mixed prior to presentation to the immune system, or presented separately.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Adjuvants can be generally divided into several groups based upon their composition. These groups include lipid micelles, oil adjuvants, mineral salts (for example, AlK(SO₄)₂, AlNa(SO₄)₂, AlNH₄(SO₄)), silica, kaolin, and certain natural substances, for example, wax D from Mycobacterium tuberculosis, substances found in Corynebacterium parvum, or Bordetella pertussis, Freund's adjuvant (DIFCO), alum adjuvant (Alhydrogel), MF-50 (Chiron) Novasomes™, or micelles, among others.

Suitable excipients for liquid formulation include water or saline, suspending agents such as sodium carboxymethylcellulose, methylcellulose, hydroxypropylmethyl-cellulose, sodium alginate, polyvinylpyrrolidone, gum tragacanth, and gum acacia; dispersing or wetting agents such as lecithin, condensation products of an alkylene oxide with fatty acids (e.g., polyoxethylene stearate), condensation products of ethylene oxide with long chain aliphatic alcohols (e.g., heptadecethyleneoxy-cetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyoxyethylene sorbitan monooleate).

Suitable excipients for solid formulations include calcium carbonate, sodium carbonate, lactose, calcium phosphate, or sodium phosphate; granulating and disintegrating agents such as maize starch, or alginic acid; binding agents such as starch, gelatin, or acacia; and lubricating agents such as magnesium stearate, stearic acids, or talc, and inert solid diluents such as calcium carbonate, calcium phosphate, or kaolin.

Other suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

Oral pharmaneutical compositions of the invention can contain one or more agents selected from the group consisting of sweetening agents, flavoring agents, coloring agents, and preserving agents in order to provide a pharmaneutically palatable preparation.

Liquid formulations according to the invention can contain one or more preservatives such as ethyl, n-propyl, or p-hydroxy benzoate; one or more coloring agents; one or more flavoring agents; or one or more sweetening agents such as sucrose, saccharin, or sodium or calcium cyclamate.

Liquid pharmaceutical formulations according to the invention, especially those comprising a phosphotidylcholine composition or an EFA composition can contain antioxidants such as tocopherol, sodium metabisulphite, butylated hydroxytoluene (BHT), butylated hydroxyanisole (BHA), ascorbic acid or sodium ascorbate.

The pharmaneutical compositions of the invention are in the form of sterile, pyrogen-free preparations suitable for parenteral administration, for example as a sterile injectable aqueous solution, a suspension or an emulsion. Such pharmaneutical compositions can be formulated using the excipients described above for liquid formulations. For example, a sterile injectable preparation according to the invention can comprise a sterile injectable solution, suspension or emulsion in a non-toxic, parenterally-acceptable diluent or solvent; e.g., as a solution in 1,3-butanediol, water or saline solution. Formulations of sterile, pyrogen-free pharmaneutical compositions suitable for parenteral administration are within the skill in the art.

3. Methods of Treating Parkinson's Disease

A subject presenting with symptoms indicative of Parkinson's Disease, or a subject at risk for developing Parkinson's Disease can be treated by the methods and compositions of the invention to prevent or delay the onset of Parkinson's Disease symptoms. The “treatment” provided need not be absolute, i.e., the Parkinson's Disease need not be totally prevented or treated, provided that there is a statistically significant improvement relative to a control population. Treatment can be limited to mitigating the severity or rapidity of onset of symptoms of the disease.

A typical regimen for preventing, suppressing, or treating a disease or condition related to Parkinson's Disease comprises administration of an effective amount of the composition as described above, administered as a single treatment, or repeated as enhancing or booster dosages, over a period up to and including one week to about 48 months or more.

The compositions of the invention can be administered to the subject by any parenteral or enteral technique suitable for introducing the composition into the blood stream or gastrointestinal tract, including intravascular (e.g., intravenous and intraarterial) injection and oral administration. In a preferred embodiment, one or more compositions are administered to the subject both by mouth, intravascularly, or both.

An “effective amount” of the compositions of the invention is any amount sufficient to therapeutically inhibit the progression of Parkinson's Disease, or to prophylactically delay the onset of Parkinson's Disease symptoms. For example, the concentration of phosphatidylcholine in a composition can range from about 500 mg to about 10,000 mg or more, about 6000 mg to about 7500 mg, from about 2000 to about 5000 mg, and from about 3000 mg to about 4000 mg phosphatidylcholine. It is intended herein that by recitation of such specified ranges, the ranges recited also include all those specific integer amounts between the recited ranges. For example, in the range of about 3000 mg to 4000 mg, it is intended to also encompass 3200 mg to 43000 mg, 3300 mg to 3800 mg, etc, without actually reciting each specific range therewith. Phosphatidylcholine compositions can be administered intravenously, orally, or both.

One of ordinary skill in the art can readily determine an appropriate temporal and interval regimen for administering the compositions of the invention. For example, the compositions of the invention can be administered once, twice or more daily, for one, two, three, four, five, six or seven days in a given a week. The length of time that the subject receives the composition can be determined by the subject's physician or other health care providers and caretakers, according to need. Due to the chronic and progressive nature of Parkinson's Disease, it is expected that subjects will receive one or more compositions according to the present methods for an indefinite period of time, likely for the rest of their lives.

In one embodiment of the invention, a phosphatidylcholine composition containing about 500 mg to 1000 mg phosphatidylcholine is administered to a subject intravenously, for example two to three times daily, for consecutive or non-consecutive days in a given week. Another phosphatidylcholine composition which contains about 3600 mg to about 18,000 mg phosphatidylcholine is administered, for example once or twice, to the same subject daily by mouth.

In one embodiment, one or more compositions comprising linoleic acid and alpha linolenic acid in an approximately 4:1 (v/v) ratio are administered to a subject who has been diagnosed with, or who is at risk for developing, Parkinson's Disease. Linoleic acid, and alpha linolenic acid, can be administered separately to a subject, as long as the ratio (v/v) of linoleic acid to alpha linolenic acid administered within a given time frame (e.g., 24 hours or less, 12 hours or less, 6 hours or less, or 4 hours or less) is approximately 4:1. The term “EFA 4:1 composition” therefore refers to one or more compositions comprising linoleic acid and one or more compositions comprising alpha linolenic acid, which are administered separately or together to a subject at about 4:1 (v/v) ratio of linoleic acid to alpha linoleic acid.

Any commercially available preparation comprising linoleic acid and alpha linolenic acid, or mixtures of the two in an approximately 4:1 (v/v) ratio, can be used as the EFA 4:1 composition in the present methods. Suitable EFA 4:1 compositions include the BodyBio Balance 4:1™ EFA oil available from BodyBio Inc. (Millville, N.J. USA), or any mixtures containing the essential fatty acids, such as for example, a mixture of cold pressed organic safflower or sunflower oil and flaxseed oil to yield a 4:1 ratio of linoleic acid to linolenic acid (4 parts Omega 6:to 1 part Omega 3).

The EFA compositions can be administered to a subject by any parenteral or enteral technique suitable for introducing the EFA composition into blood stream or the gastrointestinal tract. In a preferred embodiment, the EFA 4:1 compositions are administered to the subject by mouth.

An “effective amount” of EFA 4:1 compositions is any amount sufficient to inhibit the progression of Parkinson's Disease, or to delay the onset of Parkinson's Disease symptoms, when administered in conjunction with the phosphatidylcholine and one or more compositions containing trace minerals, rGlutathione, butyrate, electrolytes, methylating agents (folinic acid, methylcobalamin), or a combination thereof. For example, an effective amount of the EFA 4:1 composition can be from about 10 mls (about 2 teaspoons) to about 100 mls (about 7 tablespoons), about 15 mls (about 1 tablespoon) to about 80 mls (about 5 tablespoons), or about 30 mls (about 2 tablespoons) to about 60 mls (about 4 tablespoons).

One skilled in the art can readily determine an appropriate dosage regimen for administering the EFA compositions. For example, the EFA compositions can be administered once, twice or more daily, for one, two, three, four, five, six or seven days in a given week. The length of time that the subject receives EFA compositions can be determined by the subject's physician or primary caretaker, according to need. Due to the chronic and progressive nature of Parkinson's Disease, it is expected that subjects will receive EFA compositions according to the present methods for an indefinite period of time, likely for the rest of their lives.

In one embodiment, about 30 mls to about 60 mls (about 2 to about 4 tablespoons) of the EFA 4:1 compositions are administered to a subject by mouth, once to twice daily.

In another embodiment, gamma linolenic acid is administered by mouth as evening primrose oil from about 910 mg to about 2600 mg.

In the practice of the present methods, an effective amount of compositions comprising trace minerals are administered to subject who has been diagnosed with, or who is at risk for developing, Parkinson's Disease. The trace minerals in one or more same or different compositions are administered to the subject, or two or more mineral compositions can be administered separately. It is understood that mineral compositions can be administered separately to a subject, as long as the compositions are administered within a given time frame (e.g., 24 hours or less, preferably 12 hours or less, more preferably 6 hours or less, particularly preferably 4 hours or less). Preferably, mineral compositions for use in the present methods comprise biologically available forms of potassium, magnesium, zinc, copper, chromium, manganese, molybdenum, selenium, iodine, or any combination thereof, although the mineral compositions can comprise other minerals in biologically available form.

The compositions comprising trace minerals can be administered to a subject by any parenteral or enteral technique suitable for introducing the compositions into the blood stream or gastrointestinal tract. In one embodiment, the compositions comprising trace minerals are administered to the subject by mouth.

Also encompassed within the scope of the invention is the use of the electrolytes. In one embodiment, a balanced electrolyte concentrate is administered orally with one to fifteen tablespoons diluted in fluid. E-Lyte Balanced Electrolyte is a concentrated high K:Na ratio solution that is usually diluted with H₂O at 16:1. In another embodiment the subject is instructed to take the electrolyte in its concentrated form, one to three tablespoons at a time followed by 1 or 2 ounces of H₂O, throughout the day.

Any commercially available composition or compositions comprising one or more biologically available minerals can be used as trace mineral composition of the present invention. Suitable mineral compositions include solid multi-mineral preparations, or the E-Lyte Liquid Mineral™ set #1-8 (separate solutions of biologically available potassium, zinc, magnesium, copper, chromium, manganese, molybdenum, and selenium) or #1-9 (separate solutions of biologically available potassium, zinc, magnesium, copper, chromium, manganese, molybdenum, selenium and iodine), both available from E-Lyte, Inc. (Millville, N.J. USA).

The effective amount of the trace minerals is determined for each subject according to that subject's needs and nutritional status, based on a nutritional evaluation of the subject. Suitable techniques for performing a nutritional evaluation of a subject include standard blood tests to determine serum mineral and electrolyte levels, and subjective evaluations such as the E-Lyte, Inc. “taste test” for determining mineral deficiencies. The E-Lyte, Inc. “taste test” for determining mineral deficiencies is described below in the Examples.

After determining the effective amount of the one or more mineral compositions for administration to the subject, one skilled in the art can readily determine the dosage regimen for administering mineral compositions. For example, the trace minerals can be administered once, twice or more daily, for one, two, three, four, five, six or seven days in a given week. Preferably, the one or more mineral compositions are administered to the subject twice a day, for seven days in a given week. The length of time that the subject receives the mineral compositions can be determined by the subject's physician or primary caretaker, according to need. Due to the chronic and progressive nature of Parkinson's Disease, it is expected that subjects will receive the one or more mineral compositions according to the present methods for an indefinite period of time, likely for the rest of their lives.

In another embodiment, a subject being treated according to the present methods receives intravascular (e.g., intravenous) reduced Glutathione. For example, a subject can receive from about 1000 mg to about 3000 mg of rGlutathione, about 1500 mg to about 2800 mg rGlutathione, about 1800 mg to about 2400 mg rGlutathione, once, twice or more daily, for one, two, three, four, five, six or seven days a week. In one embodiment, the subject receives about 1800 mg to about 2400 mg intravenous rGlutathione twice daily, for three consecutive or non-consecutive days in a given week. In another embodiment, the rGlutathione is administered in reduced form as an intravenous “fast push” over three to five minutes.

Any commercially available composition comprising rGlutathione can be used in the present methods. Suitable compositions comprising rGlutathione include the rGlutathione preparations from Wellness Health and Pharmaceuticals (Birmingham, Ala. USA) or Medaus Pharmacy (Birmingham, Ala. USA).

It is also preferable to maintain a subject being treated by the present methods on a low carbohydrate, high protein, high green vegetable, high legume as butter beans/mucuna, high fat diet termed the Detoxx Diet, e.g., a diet excluding all grains, sugars, fruit, fruit juices, all “below ground” root vegetables and processed foods. Suitable low carbohydrate, high protein, high fat diets include such well-known diets as Atkins® or the South Beach Diet™ (see, e.g., Atkins R C, Atkins for Life, St. Martins Press, NY, 2003 and Agatston A, THE SOUTH BEACH DIET: THE DELICIOUS, DOCTOR-DESIGNED, FOOLPROOF PLAN FOR FAST AND HEALTHY WEIGHT Loss, Random House, NY, 2003, the entire disclosures of which are herein incorporated by reference). A diet lower in carbohydrate suppresses phospholipase A2 (PLA2), an enzyme that stimulates the catalyzing or breaking apart of the essential fatty acids from the phospholipids in the cell membrane, thereby de-stabilizing the membrane and control of cellular function.

Oral support with neurotransmitter precursors is helpful with the amino acids tryptophan, theonine, mucuna beans, butter beans, tyrosine, and phenylalanine as indicated by testing of urinary neurotransmitters.

In one embodiment, the subject being treated for Parkinson's Disease receives rGlutathione as well as phosphatidylcholine and Leucovorin, which are administered intravenously and methylcobalamin is administered by injection. This treatment regimen is termed the PK Protocol.

In another embodiment, the present methods comprise treating a subject who has been diagnosed with Parkinson's Disease, or who is at risk for developing Parkinson's Disease, for an indefinite period of time (e.g., five weeks or more) by:

1) intravenous administration of a phosphatidylcholine composition comprising about 500 mg to 1000 mg phosphatidylcholine, followed by intravenous administration of Leucovorin, folinic acid at about 5 mg to 10 mg, and as the third part of the infusion about 1800 mg to about 2400 mg of rGlutathione, twice to three times daily for a minimum 3 to 5 days in a seven-day period;

2) once or twice daily oral administration of a PC composition comprising about 3600 to about 7200 mg of phosphatidylcholine, twice daily oral administration of butyrate as 5 capsules twice daily of Magnesium/Calcium Butyrate in capsule form or 3 Tablespoons or about 45 mls of liquid phenylbutyrate twice daily and/or IV administration of sodium phenylbutyrate as 5 to 10 grams;

3) once daily oral administration of an effective amount of one or more mineral compositions, (the effective amount of the one or more mineral compositions can be doubled or tripled); and

4) once daily oral administration of about 30 mls to about 60 mls (about 2 to about 4 tablespoons) of an EFA 4:1 composition. (The 4:1 oil can be administered as above 2 to 4 times daily as determined by the subject's physician or primary caretaker).

Also encompassed within the scope of the invention is the use of the methods and compositions of the invention in combination with other commonly used treatments, medications, and/or surgical procedures for Parkinsons's Disease, so long as such combination therapies do not impair the empirical healthy nutrient balance of the individual, which balance has been restored and maintained by the pharmaneutical compositions of the invention. Such combination therapies include the use of the pharmaneutical compositions of the invention with any other classical treatments for Parkinson's Disease, including, for example, the use of dopamine agonists, (e,g, carbidopa/levodopa), anticholinergics, MAOB inhibitors, COMT inhibitors, among others, with or without surgery.

4. Methods of Diagnosing Parkinson's Disease

There is no blood test, brain wave test, or X-ray that can diagnose Parkinson's Disease, and the only definitive diagnosis is through postmortem microscopic evaluation of brain cells by a pathologist. Also many of the motor symptoms of Parkinson's Disease mimic other conditions commonly found in older persons. Arthritis or depression can mirror many of Parkinson's Disease symptoms as can a stroke or other neurological disorders. In addition one third of all Parkinson's Disease patients may never develop tremor.

However, one of ordinary skill in the art can readily identify Parkinson's Disease symptoms in a subject, or diagnose Parkinson's Disease in a subject. The Comprehensive Management of Parkinson's Disease, a natural history of Parkinson's Disease is well documented (Cohen M, Weiner W J, 1994 Demos). Parkinson's Disease, Diagnosis and Management, Factor S A, Weiner W J, 2002, section II, pp 31-109, Demos New York, the entire disclosure of which is herein incorporated by reference). The presenting symptoms of Parkinson's Disease include, resting tremor: a rhythmic oscillation of a body part, such as a hand shaking back and forth with extension and flexion of the wrist; rigidity: a persistent and relatively constant tightening and stiffening of muscles that can be felt by an examiner and sensed by a patient as muscle stiffness; bradykinesia: slowness in voluntary movements, among other symptoms.

An accurate diagnosis will usually contain at least two of the three aforementioned symptoms, tremor, rigidity, and bradykinesia, however, it is important for the physician look for objective signs of Parkinson's Disease on physical examination. These signs include an obvious tremor, muscle rigidity, and imbalance that would not be caused by depression. Normal aging involves a gradual slowing down of both thought and motion, often coupled with changes in posture, memory, arthritis, and balance, which are further complicated from the influence of a variety of medications.

Rarely, some Parkinson's Disease patients are misdiagnosed initially as having a stroke. The typical signs and symptoms of a stroke can vary, but may involve weakness and stiffness on one side of the body. Parkinson's Disease often involves similar symptoms of severe rigidity and bradykinesia, usually worse on one side, which a physician might perceive as representing a stroke. Stroke symptoms almost always develop quickly over several minutes or hours that are much different from Parkinson's Disease, which progresses over many years. Further the brain scan in Parkinson's Disease is normal in appearance, whereas, after two days of onset, the scan of a stroke victim shows a dark spot on a CAT scan or a bright spot on an MRI. Additionally, if a stroke has occurred, there will be no improvement with medication (Gershanik O, “Parkinson's Disease” In Tolosa E, Koller W C, et al., Differential Diagnosis and Treatment of Movement Disorders, Boston: Butterworth-Heinemann, 1998; pp. 7-25).

5. Test Kits

The invention also provides a pharmaneutical pack or kit comprising one or more containers filled with one or more compositions or the ingredients of the pharmaneutical compositions of the invention. The kits are provided for the treatment of Parkinson's Disease or for delaying the onset of Parkinson's Disease symptoms. The kit comprises instructions for treating Parkinson's Disease in a subject, or for delaying the onset of Parkinson's Disease symptoms in a subject, and one or more of the following components: 1) a phosphatidylcholine composition; 2) an EFA 4:1 composition; 3) mineral compositions, 4) electrolyte compositions; 5) methylating agents, methylcobalamin and folinic acid/Leucovorin; 6) rGlutathione; 7) butyrate or phenylbutyrate, or a combination thereof.

If a particular component is not included in the kit, the kit can optionally comprise information on where to obtain the missing component, for example an order form or uniform resource locator for the internet specifying a website where the component can be obtained.

The instructions provided with the kit describe the practice of the methods of the invention as described above, and the route of administration and effective concentration and the dosing regimen for each of the compositions provided therein.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims. The contents of all references, patents and published patent applications cited throughout this application are expressly incorporated herein by reference.

### EXAMPLES

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### Example 1

### Parkinson's Patients Case Studies

Case Study 1

Female patient age 77 was diagnosed with Parkinson's Disease in March 2002. Patient presented with gait disturbance, unable to dance, weakness, frequent falls, frozen facies, tremor in upper extremities, left greater than right. Patient began oral nutrient supplementation with nutrient dense, low carbohydrate diet. IV therapy commenced with Glutathione push once weekly whereby after 6 months patient felt that she was stronger and her tremor was slightly improved but no other apparent change. IV PC was added to the patient's therapy once weekly. After 8 infusions patient had a dramatic response to therapy as tremor was completely resolved, gait normalized, facial expression returned, movement was organized and fluid. Patient's red cell lipids were tested in March and re-tested in December. The results demonstrated that the suppression of myelination markers was normalized. All symptoms of Parkinson's have cleared. Patient continues the diet, supplements and weekly IV infusions of PC with rGlutathione for longevity purposes. Two years later, the patient received weekly infusions of Leucovorin with IV therapy and remarkable progress was noted with more fluid movement. Patient continues with daily oral high dose essential fatty acid and nutrient therapy, low carb diet and weekly infusions of PC, Leucovorin and rGlutathione enjoying a normal lifestyle.

Case Study 2

Male patient age 51 was diagnosed with Parkinson's Disease December 2002. Patient presented with tremor in left arm, muscle stiffness, abnormal gait, muscle weakness, sound sensitivity and poor carbohydrate tolerance. Patient began oral nutrient supplementation with nutrient dense diet after 3 months from the onset of the disease. After 2 weeks of IV therapy with PC and Glutathione push, a total of 20 infusions, patient's tremor was 30% improved and his gait became more fluid.

Case Study 3

Female patient age 60 was diagnosed with Parkinson's Disease in May 1995. Patient presented with resting tremor, fatigue, muscle pain/weakness/spasticity/spasm, slow movement, small shuffling steps, reduced arm swing, frozen facies, dry skin, cramping in right leg, insomnia, irritability, apathy, abnormal gait, joint pain, aphasia, sciatica. After 3 weeks of oral nutrients and IV PC and glutathione therapy patient's tremor was 50% improved, fatigue, muscle spasm and spasticity were much improved and gait was smoother. Facial expression was improved and patient was able to express herself with more ease and comfort.

Case Study 4

Male patient age 65 was diagnosed with Parkinson's Disease August 2004. Patient presented with resting tremor in right arm/leg, poor coordination, abnormal gait, aphasia, anxiety, chronic fatigue, frozen facies, rigidity, muscle cramps, small handwriting, reduced arm swing, small shuffling steps, insomnia, joint pain, light sensitivity, history of kidney stones and cataracts. After 1 week of oral nutrient and IV therapy patient's tremor was 30% improved, fatigue, muscle spasm, and rigidity were much improved and gait was more fluid. Facial expression was improved and patient became happier and more talkative with a sparkle in his eyes.

Case Study 5

Male patient age 52 was diagnosed with Parkinson's Disease in November 2001. Patient presented with right side tremor impacting both arm and leg (drags right leg), muscle stiffness in right arm and leg, abnormal gait, poor coordination, shakiness, difficulty swallowing, vertigo, poor memory, brain fog, urinary frequency, aphasia, frozen facies, vertigo, psoriasis, dry skin, bad breath and has a history of panic disorder. After 2 weeks of oral nutrient and IV PC, Leucovorin and Glutathione therapy patient's tremor was 20% improved, stiffness in the arm was improved, his energy was increased, his gait was faster and smoother and there were no longer dragging of the right leg, his thinking was clearer, his mood improved, he was laughing more and his facial expression was more fluid.

Case Study 6

Male patient age 41 was diagnosed with Parkinson's Disease in February 2002. Patient presented with resting tremor right arm and jaw, severe fatigue, muscle pain/rigidity/atrophy in right arm, slow movement, abnormal gait, frozen facies, dry skin, insomnia, nervousness, fasciculations, apathy, urinary urgency, brain fog, seborrheic dermatitis, nausea, and Lyme Disease. Trials with Parkinson's drugs were unsuccessful and patient was intolerant to L-Dopa. After 1 week of oral nutrient therapy and four infusions of PC, Leucovorin and Glutathione therapy patient's tremor was 5% improved, his fatigue was lessened, facial expression was softer, there were more clarity of thought, and fewer fasciculations with smoother gait.

Case Study 7

Male patient age 85 was diagnosed with Parkinson's Disease in August 2004.,Patient presented with frozen facies, severe fatigue, slow movement, stiff gait, freezing when attempting to walk, poor memory, dry skin, insomnia, apathy, abnormal gait, severe back (scoliosis) and joint pain, aphasia, depression, anxiety, tan stool, dry skin, reflux, dementia and family history of Parkinson's. After 3 months of oral nutrient therapy and IV PC and Glutathione once weekly patient's memory, frozen facies, tan stool, apathy, fatigue, stiff gait and dry skin was much improved while dementia was slightly improved. IV Leucovorin was introduced as 20 mg and oral leucovorin at 16 mg. After one week of adding leucovorin into the IV protocol and oral leucovorin was added patient had a dramatic change in alertness and memory and his depression, insomnia and dementia were dramatically improved. Gait was smoother and facial expression was much more expressive. Patient continues to improve with weekly infusions of PC, Leucovorin and Glutathione along with oral nutrient therapy.

### Example 2

### Testing for E-Lyte Liquid Minerals

The test determines mineral deficiency using a taste test for 8 different minerals. 1. potassium phosphate, 2. zinc sulphate, 3. magnesium chloride, 4. copper sulfate 5. potassium chromate, 6. potassium per-manganate or manganese gluconate, 7. ammonium molybdate, and 8. selenium selenite. Number 9, potassium iodide, is not included in the taste test protocol but is included in the daily mineral drink, however, no more than one portion per day.

To test the 8 liquid minerals, a portion of each mineral is pored in a small cup starting with #1. Using about 2-3 teaspoons, each liquid mineral is placed in the mouth and swished to effectively obtain a taste response. Check the score card below and pick a number that matches the taste response. Mark down the score and proceed to the next until all 8 minerals are done.

Taste Test Score

Sweet

Pleasant

No Taste

Hmmm . . . Taste Something

So . . . So

Don't Like

Awful

A score of 1 or 2 indicates deficiency with a 1 being quite deficient. A score of 3 indicates need, while a score of 4 indicates sufficiency. If the taste sensation is neither pleasant nor disturbing but is clearly not just plain water, it is a 4, and indicates a lack of need at this time. (4 is the ultimate goal). A 5, while not unpleasant, could be avoided, while a score of 6 or 7 indicates an excess of that mineral and should be avoided at this time.

Minerals tasted between 1 and 4 should be taken, together or individually, with liquids, such as, for example, and an acidic juice (orange, grapefruit, or pineapple), or ¼ tsp of vitamin C powder. The body requirement for the type and the concentration of minerals changes frequently, therefore frequent testing of the liquid minerals is important. Taking the minerals that is approved by the mineral testing shifts the body into a balanced state which is the ultimate goal.

### Example 3

### Intravenous Administration of The Pharmaneutical Compositions

a) Administration of PC Composition

A butterfly catheter with a 23-gauge needle was inserted into a vein of the antecubital region of one of the subjects' arms. A syringe containing the PC (phosphatidylcholine) composition in about 5 to 20 cc volume was connected to the catheter by a flexible tube. A volume of blood equal to the total volume of the PC composition was drawn into the syringe and the syringe was gently agitated to mix the blood and PC composition. The blood/PC composition mixture was then infused (or “pushed”) as a lipid exchange into the subject over a period of two to three minutes.

b) Intravenous Administration of Leucovorin, Folinic Acid as Tetrahydrofolate

A butterfly catheter with a 23-gauge needle was inserted into a vein of the antecubital region of one of the subjects' arms. The PC composition was infused first followed by a pre-prepared syringe containing about 5 mg (0.5 cc) to 10 mg (1 cc) of Leucovorin over the period of 2-3 minutes.

c) Intravenous Administration of Reduced Glutathione

A butterfly catheter with a 23-gauge needle was inserted into a vein of the antecubital region of one of the subjects' arms. The PC and Leucovorin compositions were infused first followed by a pre-prepared syringe containing about 9 to 15 cc of glutathione generally pre-mixed with an equal portion of sterile water (not saline). The composition containing glutathione was followed the IV PC with a pre-prepared syringe of glutathione using the same needle. This procedure avoids re-sticking the patient by infusing first the PC, then the Leucovorin and then the glutathione using the same butterfly catheter with a flexible tube infused (or “pushed”) into the subject over a period of two to five minutes.

### Example 4

### Treatment of Parkinson's Disease Using Pharmaneutical Compositions

Twenty subjects diagnosed with Parkinson's Disease (see Table 1) were treated according to the protocol outlined below for at least five weeks, and were evaluated daily for any improvement in Parkinson's Disease symptoms. The subjects were kept on a low carbohydrate, high protein, high fat diet (e.g., a diet excluding all grains, sugars, fruit, fruit juices and all “below ground” root vegetables).

TABLE 1
Clinical Characteristics of Subjects
Diagnosed with Parkinson's Disease

| | | | Age at | |
|---|---|---|---|---|
| Subject | | | PD | |
| No. | Age | Sex | onset | Symptoms at start of protocol |
| 1 | 77 | F | 77 | Gait Disturbance, Resting tremor, |
| | | | | Frozen Facies, Frequent Falls |
| 2 | 51 | M | 51 | Gait Disturbance, Resting tremor, |
| | | | | Frozen Facies, Weakness |
| 3 | 60 | F | 52 | Gait Disturbance, Resting tremor, |
| | | | | Frozen Facies, Fatigue |

1) intravenous administration of 500 mg to 1000 mg Essentiale N™ or LipoStabil™ phosphatidylcholine (A. Natterman & Cie, GmbH, Cologne, Germany), followed by intravenous administration of 1800 mg to 2400 mg of reduced glutathione, twice daily for 3 days in a seven-day period;

2) once daily oral administration of ten to twenty capsules (900 mg phosphatidylcholine each) of Nutrasal™ PC (Nutrasal LLC, Oxford, Conn. USA) or E-Lyte PhosChol™ (E-Lyte, Inc., Millville, N.J. USA); once or twice daily oral administration of butyrate of five capsules. (E-Lyte, Inc. Millville, N.J. USA).

3) once or twice daily oral administration of triple portions of various minerals from the E-Lyte Liquid Mineral™ set #1-8 (E-Lyte, Inc., Millville, N.J. USA), as determined by the E-Lyte mineral taste test protocol described above; and

4) once or twice daily oral administration of 30 mls to 60 mls (about 2 to about 4 tablespoons) BodyBio Balance 4: 1™ EFAs (E-Lyte, Inc., Millville, N.J. USA).

Subject 1 was diagnosed with Parkinson's Disease in March of 2002, and presented with tremor, masked facies, and abnormal gait. By the time the above treatment protocol was fully initiated in September through November of 2002 with the use of BodyBio Balance 4:1™ EFAs, rGlutathione infusions and oral therapy with Electrolytes, Liquid Trace Minerals, and Butyrate, all the patient's symptoms had resolved. Patient continues to be symptom free as up-to-date (May 2005).

All references discussed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

We claim:

1. A pharmaneutical composition for treating, preventing, or ameliorating the symptoms of Parkinson's Disease comprising an effective amount of a first and a second composition, the first composition comprises one or more phosphatidylcholine formulations and the second composition comprises one or more constituents comprising essential fatty acid supplements, trace minerals, butyrate, electrolytes, methylating agents, glutathione, or a combination thereof, in a suitable carrier.

2. The pharmaneutical composition of claim 1, wherein the first composition, the second composition, or both are formulated in one solution.

3. The pharmaneutical composition of claim 1, wherein the first composition, the second composition, or both are formulated in different solutions.

4. The pharmaneutical composition of claim 1, wherein the first composition, the second composition, or both are administered contemporaneously.

5. The pharmaneutical composition of claim 1, wherein the first composition, the second composition, or both are administered at different time intervals.

6. The pharmaneutical composition of claim 1, wherein the first composition, the second composition, or both are administered in a time-released manner.

7. The pharmaneutical composition of claim 1, wherein the first composition, the second composition, or both are in a dry formulation.

8. The pharmaneutical composition of claim 7, wherein the first composition, the second composition, or both are in a liquid formulation.

9. The pharmaneutical composition of claim 1, wherein the essential fatty acid supplements comprise linoleic acid and alpha linolenic acid in a ratio of about 4:1.

10. The pharmaceutical composition of claim 1, wherein the methylating agents comprise vitamin B compounds.

11. The pharmaneutical composition of claim 10, wherein the vitamin B compounds comprise B12, and B complex compounds.

12. The pharmaceutical composition of claim 11, wherein the B12 and B complex compounds comprise Methylcobalamin, and folinic acid compounds comprising Leucovorin, Citrovorum and, Wellcovorin, or a combination thereof.

13. The pharmaneutical composition of claim 1, wherein the trace minerals comprise E-Lyte Liquid Mineral™ set #1-8 containing separate solutions of biologically available potassium, zinc, magnesium, copper, chromium, manganese, molybdenum, and selenium, or a combination thereof.

14. The pharmaneutical composition of claim 1, wherein the electrolytes comprise sodium, potassium, chloride, calcium, magnesium, bicarbonate, phosphate, and sulfate, or a combination thereof.

15. A method of treating, ameliorating, or preventing the symptoms of Parkinson's Disease in a subject comprising administering to the subject an effective amount of a pharmanuetical composition comprising a first and a second composition, the first composition comprising one or more phosphotidylcholine formulations and the second composition comprises one or more constituents comprising essential fatty acid supplements, trace minerals, butyrate, electrolytes, methylating agents, glutathione, or a combination thereof, in a suitable carrier, wherein the subject is treated or the symptoms of Parkinson's Disease in the subject are ameliorated, or prevented.

16. The method of claim 16, wherein the first composition, the second composition, or both are administered intravenously, orally, or both.

17. The method of claim 16, wherein the one or more phosphotidylcholine formulation comprise intravenous and oral formulations.

18. The method of claim 17, wherein about 500 mg phosphatidylcholine is administered to the subject intravenously twice daily for about three days a week, and about 3600 mg to about 7200 mg phosphatidylcholine is administered to the subject daily by mouth.

19. The method of claim 1, wherein about 30 mls to about 60 mls of the EFA 4:1 is administered to the subject daily by mouth.

20. The method of claim 1, wherein the trace minerals comprise a biologically available form of sodium, potassium, magnesium, zinc, copper, chromium, manganese, molybdenum, selenium, iodine, or any combination thereof.

21. The method of claim 1, wherein the trace minerals are administered to the subject up to three times daily.

22. The method of claim 1, wherein the reduced glutathione is administered intravenously at about 1800 mg to about 2400 mg, 1-3 times daily, and for 2-4 days in a seven-day period.

23. The method of claim 1, wherein the subject is maintained on a low carbohydrate, high protein, high fat diet.

24. A method of treating, ameliorating, or preventing the symptoms of Parkinson's Disease in a subject comprising:
i) intravenous administration of a first phosphatidylcholine composition comprising about 500 mg to 1000 mg phosphatidylcholine, followed by intravenous administration leucovorin of about 5 mg to about 10 mg, and followed by about 1800 mg to about 2400 mg of reduced glutathione, twice daily for 3 to 5 days in a seven-day period;
ii) once daily oral administration of a second phosphatidylcholine composition comprising about 3600 to about 18,000 mg of phosphatidylcholine daily;
iii) once or twice daily oral administration of an effective amount of one or more trace minerals;
iv) five times daily oral administration of electrolytes;
v) once or twice daily oral administration of about 30 mls to about 60 mls of an EFA 4:1 composition;
vi) once or twice daily oral administration of about 910 mg to about 2600 mg gamma linolenic acid as evening primrose oil;
vii) once or twice daily oral or intravenous administration of an effective amount of one or more vitamin B complex compositions, Leucovorin/Folinic acid; and
viii) once daily oral, sublingual, or injectable administration of an effective amount of one or more Methylcobalamin compositions,
wherein the subject is treated or the symptoms of Parkinson's Disease in the subject are treated, ameliorated, or prevented.

25. A kit for the treatment, amelioration, or prevention of the symptoms of Parkinson's Disease in a subject, comprising:
a) a first composition comprising one or more phosphatidylcholine formulations;
b) a second composition comprising one or more constituents comprising:
i) linoleic acid and alpha linolenic acid in a ratio of about 4:1;
ii) trace minerals;
iii) butyrate or phenylbutyrate;
iv) electrolytes;
v) methylating agents; and
vi) glutathione,
c) instructions for the use of the first and second compositions; and
d) instructions for where to obtain any missing components of the kit.

26. The kit of claim 22, further comprising instructions for determining an effective amount of the trace minerals for administration to the subject.

27. The kit of claim 22, wherein the first composition, the second composition, or both are formulated in one or different solutions.

28. The kit of claim 22, wherein the one or more constituents are formulated in one or different solutions.

29. The kit of claim 22, wherein the first composition, the second composition, or both are in a liquid or dry formulation.

30. The kit of claim 22, wherein the methylating agents comprise Methylcobalamin, folinic acid, Leucovorin, or a combination thereof.
